(19) United States
(12) Patent Application Publication
(10) Pub. No.: US 2025/0222064 A1
(43) Pub. Date: Jul. 10, 2025

(54) METHOD OF TREATMENT OF NEUTROPHIL-DRIVEN INFLAMMATORY PATHOLOGIES

(71) Applicant: WILD BOAR BIOSCIENCES, LLC, Tempe, AZ (US)

(72) Inventors: Laura L. Eggink, Scottsdale, AZ (US); J. Kenneth Hoober, Phoenix, AZ (US)

(21) Appl. No.: 18/850,536

(22) PCT Filed: Nov. 8, 2022
(86) PCT No.: PCT/US2022/079511

§ 371 Date: Sep. 24, 2024

Related U.S. Application Data
(60) Provisional application No. 63/323,495, filed on Mar. 24, 2022.

(51) Int. Cl. A61K 38/10; A61K 9/00; A61K 47/64; A61P 17/06

(52) U.S. Cl. CPC A61K 38/10; A61K 9/0014; A61K 47/64; A61P 17/06

(57) ABSTRACT

The present invention provides a method of treating pathological inflammation in a patient comprising: administering to the patient a multivalent structured polypeptide comprising at least one therapeutic peptide. The present invention also provides a kit, comprising: a multivalent structured polypeptide comprising at least one therapeutic peptide; and instructions teaching administration of the multivalent structured polypeptide to a patient having atopic dermatitis.

### CROSS-REFERENCE TO RELATED APPLICATIONS

This application claims the benefit of U.S. Provisional Application No.63/323,495, entitled “Method of Treatment of Neutrophil-Driven Inflammatory Pathologies” which was filed Mar.24, 2022, the entire disclosure of which is hereby incorporated herein by this reference.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 11,058 byte ASCII (text) file named “24667.048WO-PCT_SeqList” created on Nov.8, 2022.

### TECHNICAL FIELD

This invention relates to the treatment of inflammatory diseases of epithelial tissues caused by excessive neutrophil infiltration, such as atopic dermatitis (eczema), psoriasis, asthma and pyoderma gangrenosum with therapeutic peptides.

### BACKGROUND

This document describes a unique and effective treatment for neutrophil-driven inflammatory diseases, for example, atopic dermatitis (AD), eczema, ichthyosis vulgaris, psoriasis, pyoderma gangrenosum and asthma.

Atopic dermatitis (AD, eczema) is one of the most common inflammatory disorders affecting up to 20% of children, with age of onset 3 to 5 months, and 3 to 10% of adults (Langan et al., 2020; Bitton et al., 2020). The incidence varies between countries, with 14% in the US and 2.1% in Japan but 20% in Sweden (Shaw et al., 2011; Urban et al., 2021). More than 230 million individuals worldwide experience eczema. Approximately 2% (>125 million people) of the world population is affected by psoriasis, an immunogenic disease that in severe cases affects more than 10% of the body (Lowes et al., 2014; Chiang et al., 2019). The strongest genetic risk factors in AD are mutations in the gene encoding filaggrin, although only 20 to 40% of patients have FLG loss-of-function mutations (Smith et al., 2006). Other genetic and environmental factors account for the majority of the cases. In contrast to AD, psoriasis appears later in life, usually early adulthood, and does not improve with age (Guttman-Yassky et al., 2011). The impact on the quality of life of patients and their families by these diseases is profound and multifaceted.

Front-line treatments for AD, particularly for children, include topical corticosteroids and moisturizers to reduce inflammation. Kleinman et al. (2022) concluded from their review of the literature that with the ready availability of topical corticosteroids and inhibitors of calcineurin, phosphodiesterase 4 and Janus kinases, there still persists an “unmet need for topical therapies with high efficacy and low risk of adverse effects with long-term use.” The currently heavily advertised JAK inhibitors such as Cibingo, Rinvoq, and Opzelura, and many versions in development, were initially developed as topical drugs but also have been incorporated into oral formulations. Efficacy scoring (e.g., EASI-75) showed significant improvement in 40 to 50% of patients after 8 weeks of daily treatment. Of concern with these treatments is the fact that these drugs are immunosuppressive and are associated with risk factors for infections and malignancies when taken orally (systemically).

A type-2 immune response, with IL-4 and IL-13 as dominant factors, is a primary driver of inflammation. Dupilumab (Dupixent), a monoclonal antibody, is widely used to treat moderate-to-severe eczema, particularly patients who are refractory to topical agents. The recommended dose is 300 mg of the antibody injected subcutaneously every two weeks. Dupilumab binds to IL-4Ra, the common subunit of the receptors for IL-4 and IL-13, which mediates TH2 differentiation and pro-allergic adaptive immune responses and subdues the inflammation mediated by IL-4/IL-13. Although an immunosuppressive drug, as the topical drugs indicated above, the greater specificity allows longer-term treatment that extends to a year or more. About 65% of treated patients experience an EASI-75 at week 16, with the remaining population being refractory. Whereas effects are detected as early as after two weeks of treatment, the maximal response is not achieved until about 16 weeks of treatment. Although less frequent in clinical trials, in the real world a common side-effect is conjunctivitis, which may occur in nearly two-thirds of patients in the real world (Kamata and Tada, 2021). Dupilumab generally has a more favorable safety profile than JAK inhibitors, and subsequent healing of the surface barrier reduces cutaneous infections. The costs and logistics of production and distribution are significant but generally covered by individual insurance programs (Kamata and Tada, 2021).

Several types of immune cells play a role in psoriasis, with IL-23 produced by CD301b⁺dendritic cells (DCs) playing a pivotal role in stimulating IL-17 production by activated T cells (Lowes et al., 2014; Kim et al., 2018). Munera-Campos and Carrascosa (2019) and Saini and Pansare (2019) listed the many additional antibodies against IL-13, IL-22, IL-33 and other cytokines that are under development and the large number of small molecules being developed for topical or oral applications.

Populations that are refractory to these treatments still represent large numbers of patients. Efficacy data from clinical trials benefit from the most careful treatment conditions and thus are expected to be better than in the real world. Some individuals may resist being injected with the large bolus of protein, which can be done at home by the patient but more likely by a health professional. The immunosuppressive drugs require weeks to reach maximal efficacy. Moreover, the side-effects often preclude continuation of the treatment. Clearly, as Kleinman et al. (2022) state, the need exists for improved and less-toxic treatments for atopic dermatitis.

SBI's treatment takes advantage of the natural healing efforts of the skin to repair the surface barrier and ameliorate the symptoms. The initial inflammatory response to injury induces expression of transglutaminases (TGMs) in keratinocytes. TGM2 is released from these cells and catalyzes formation of cross-links between corneocytes to tighten the loose surface barrier. However, the paucity of available glutamine residues in proteins on the surface of corneocytes for this reaction precludes rapid healing. Topical application of tetravalent, glutamine-containing peptides provides the substrate for TGMs, particularly extracellular TGM2, to cross-link corneocytes to repair the surface barrier. Restoration of the surface barrier allows keratinocytes to regain the normal differentiation program, which eliminates production of cytokines that attract neutrophils. CD301+ (CD301b in the mouse) macrophages in the dermis are also activated to phagocytize and remove residual neutrophils (Greenlee-Wacker, 2016). In studies with mouse models, normal skin morphology is restored within 2 weeks. The peptides are not toxic and are expected to be rapidly degraded without adverse effects. A combination of SBI's peptides with the drugs described above would provide a more rapid response without contributing additional toxicity. SBI's technology provides an opportunity to provide a low-cost, highly effective, non-toxic treatment for the world.

The current activity to achieve effective treatments is an indication of the need for therapies specifically against AD and psoriasis, and more serious but rare diseases, that will decrease the global burden in health care costs and morbidity caused by this malady. Thus, there is a need for a more effective treatment approach to AD, particularly for young children.

### SUMMARY

The present invention provides a two-fold pincer movement against the AD, psoriasis, and similar disorders. On the one hand, the peptides are substrates for transglutaminase-2 (TGM2) that allows formation of cross-links to restore a functional surface barrier. Secondarily, immune cells in the skin are activated to eliminate the cells that cause inflammation. The predominant immune cell type in the dermis is the CD301b⁺ macrophage. Whereas the responses of these cells to insults, whether, for example, AD or a wound, are complex, an unmet need is the ability to manipulate these cells. Macrophage responses can be targeted by ligands of the endocytic receptor CD301b (CLEC10A in humans). The receptor is specific for binding N-acetylgalactosamine (GalNAc) and thus constructs of the sugar or characterized peptide mimetics such as svL4 (SEQ ID NO: 1) and sv6D (SEQ ID NO:2) (Eggink et al., 2018) are appropriate molecules to engage the cells. To achieve specific responses, a variety of molecules can be conjugated to these targeting moieties, such as proteins, drugs, or RNA molecules (siRNA or mRNA). To protect the RNA molecules when applied topically, it may be necessary to encapsulate them into lipid-based nanoparticles. The application of the present invention can be used for treatment of many epithelial maladies.

In some aspects, the present invention relates to a method of treating a patient having a neutrophil-driven inflammatory disease, the method comprising: administering to the patient a multivalent structured polypeptide comprising at least one therapeutic peptide; wherein the sequence of the therapeutic peptide consists of the sequence X₁-X₂-X₃-X₄-X₅-X₆-X₇-X₈-X₉-X₁₀-X₁₁-X₁₂ (SEQ ID NO: 9) with each of X₁, X₂, X₃, X₄, X₅, X₆, X₇, X₈, X₉, X₁₀, X₁₁, and X₁₂ independently being absent or any amino acid residue, so long as the therapeutic peptide comprises at least 4 amino acid residues and at least one of X₁, X₂, X₃, X₄, X₅, X₆, X₇, X₈, X₉, X₁₀, X₁₁, and X₁₂ is Q.

In other aspects, the multivalent structured polypeptide has a central framework, a linker sequence, and at least two arms, wherein each arm comprises the therapeutic peptide, and each arm is linked to the central framework via the linker sequence. In one aspect, the linker sequence is selected from the group consisting of: GGGS (SEQ ID NO:8), GGGSGGGS (SEQ ID NO:9), SSSS (SEQ ID NO:10), and SSSSSSSS (SEQ ID NO:11). In another aspect, the at least one therapeutic peptide is VQATQSNQHTPR (SEQ ID NO:5), NQHTPR (SEQ ID NO:6), VQATQS (SEQ ID NO:7), or a combination thereof.

In certain aspects, the multivalent structured polypeptide is tetravalent. In one aspect, the multivalent structured polypeptide is svL4 (SEQ ID NO:1) (SEQ ID NO:1), sv6D (SEQ ID NO:2) (SEQ ID NO:2), or svC1 (SEQ ID NO:3) (SEQ ID NO:3).

In other aspects, the at least one therapeutic peptide acts as a substrate for a transglutaminase to induce cross-linking of the stratum corneum, restore the epidermal barrier, and protect the patient from environmental pathogens and allergens. In one aspect, the multivalent structured polypeptide is effective in deleting neutrophils from the dermis of inflamed skin.

In some aspects, the multivalent structured polypeptide is formulated with a pharmaceutically acceptable excipient selected from the group consisting of a preservative, a lubricant, a suspending agent, a wetting agent, a thickening agent, a biocompatible solvent, a surfactant, a complexation agent, and any combination thereof. In one aspect, the multivalent structured polypeptide is administered as a topical formulation. In another aspect, the multivalent structured polypeptide is embedded in a hydrogel.

In certain aspects, the multivalent structured polypeptide is administered with a topical corticosteroid or monoclonal antibody. In some aspects, the at least one topical corticosteroid is selected from the group consisting of triamcinolone acetonide, hydrocortisone, and a combination thereof. In other aspects, the multivalent structured polypeptide is administered by subcutaneous injection. In some aspects, the at least one monoclonal antibody is selected from the group consisting of dupilumab, nemolizumab, secukinumab, and combinations thereof.

In some aspects, the multivalent structured polypeptide and topical corticosteroid or monoclonal antibody are conjugated by a chemical conjugation or an enzymatic conjugation. In one aspect, the chemical conjugation comprises lysine amide coupling or cysteine-based conjugation, and the enzymatic conjugation comprises transpeptidation using sortase, transpeptidation using microbial transglutaminase, or N-Glycan engineering. In another aspect, the multivalent structured polypeptide is conjugated to the topical corticosteroid or monoclonal antibody via a linker comprising an activated carboxylic acid ester. In another aspect, the multivalent structured polypeptide and topical corticosteroid or monoclonal antibody are linked with biotin-avidin.

In some aspects, the method further comprises identifying the patient as having a neutrophil-driven inflammatory disease. In one aspect, the neutrophil-driven inflammatory disease is atopic dermatitis (AD), psoriasis, or asthma. In another aspect, the neutrophil-driven inflammatory disease is pyoderma gangrenosum.

In other aspects, the present invention relates to a method of treating a patient having a neutrophil-driven inflammatory disease, the method comprising: subcutaneously administering to the patient a multivalent structured polypeptide comprising at least one therapeutic peptide; wherein the sequence of the therapeutic peptide consists of NPSHPLSG (SEQ ID NO: 12).

In some aspects, the multivalent structured polypeptide has a central framework, a linker sequence, and at least two arms, wherein each arm comprises the therapeutic peptide, and each arm is linked to the central framework via the linker sequence. In one aspect, the linker sequence is selected from the group consisting of: GGGS (SEQ ID NO:8), GGGSGGGS (SEQ ID NO:9), SSSS (SEQ ID NO:10), and SSSSSSSS (SEQ ID NO:11). In another aspect, the multivalent structured polypeptide is tetravalent. In another aspect, the multivalent structured polypeptide is svHIC (SEQ ID NO:4) (SEQ ID NO:4).

In yet other aspects, the present invention relates to a kit, comprising: a multivalent structured polypeptide comprising at least one therapeutic peptide; wherein the sequence of the therapeutic peptide consists of the sequence X₁-X₂-X₃-X₄-X₅-X₆-X₇-X₈-X₉-X₁₀-X₁₁-X₁₂ (SEQ ID NO: 9) with each of X₁, X₂, X₃, X₄, X₅, X₆, X₇, X₈, X₉, X₁₀, X₁₁, and X₁₂ independently being absent or any amino acid residue, so long as the therapeutic peptide comprises at least 4 amino acid residues and at least one of X₁, X₂, X₃, X₄, X₅, X₆, X₇, X₈, X₉, X₁₀, X₁₁, and X₁₂ is Q; and instructions teaching administration of the multivalent structured polypeptide to a patient having a neutrophil-driven inflammatory disease.

In some aspects, the multivalent structured polypeptide is svL4 (SEQ ID NO:1) (SEQ ID NO: 1), sv6D (SEQ ID NO:2) (SEQ ID NO:2), or svC1 (SEQ ID NO:3) (SEQ ID NO:3). In one aspect, the kit further comprises at least one topical corticosteroid and/or at least one monoclonal antibody. In some aspects, the at least one topical corticosteroid is selected from the group consisting of triamcinolone acetonide, hydrocortisone, prednisone, and a combination thereof; and/or the at least one monoclonal antibody is selected from the group consisting of dupilumab, nemolizumab, secukinumab, and combinations thereof.

Some kits additionally comprise N-acetylgalactosamine (GalNAc) and/or a GalNac mimetic for use as described herein.

In yet other aspects, the present invention relates to a pharmaceutical composition comprising: i) a multivalent structured polypeptide comprising at least one therapeutic peptide; wherein the sequence of the therapeutic peptide consists of the sequence X₁-X₂-X₃-X₄-X₅-X₆-X₇-X₈-X₉-X₁₀-X₁₁-X₁₂ (SEQ ID NO: 9) with each of X₁, X₂, X₃, X₄, X₅, X₆, X₇, X₈, X₉, X₁₀, X₁₁, and X₁₂ independently being absent or any amino acid residue, so long as the therapeutic peptide comprises at least 4 amino acid residues and at least one of X₁, X₂, X₃, X₄, X₅, X₆, X₇, X₈, X₉, X₁₀, X₁₁, and X₁₂ is Q; and ii) either at least one topical corticosteroid selected from the group consisting of triamcinolone acetonide, hydrocortisone, and a combination thereof; or at least one monoclonal antibody selected from the group consisting of dupilumab, nemolizumab, secukinumab, and combinations thereof.

In another aspect, the present invention relates to a method of targeting a payload to a cell expressing an endocytic receptor of CD301b (also known as CLEC10A), the method comprising:
conjugating the payload to N-acetylgalactosamine (GalNAc) and/or a GalNac mimetic to form a conjugate; and contacting the cell with the conjugate; wherein the payload is a protein, drug, or RNA molecule.

In some aspects, the cell is a macrophage or a dendritic cell. In one aspect, a GalNac mimetic is conjugated to the payload. In another aspect, the GalNac mimetic is svL4 (SEQ ID NO: 1), sv6D (SEQ ID NO:2), or a combination thereof.

In some aspects, the payload is conjugated to the GalNAc and/or the GalNac mimetic by a chemical conjugation or an enzymatic conjugation. In one aspect, the payload is an RNA molecule selected from the group consisting of an mRNA, siRNA, and miRNA. In another aspect, the conjugate is encapsulated in a lipid-based nanoparticle.

In other aspects, the present invention relates to a method of treating an epithelial disease in a subject in need thereof, the method comprising administering an effective amount of a conjugate to the subject, wherein the conjugate comprises: a payload selected from the group consisting of a protein, drug, and RNA molecule; and (GalNAc) or a GalNac mimetic conjugated to the payload by a chemical conjugation or an enzymatic conjugation.

In one aspect, a GalNac mimetic is conjugated to the payload and the GalNac mimetic is sVL4 (SEQ ID NO:1), sv6D (SEQ ID NO:2), or a combination thereof.

In another aspect, the epithelial disease is a corneal disease, a conjunctival disease, an oral disease, an epidermal disease, or a hyperproliferative epithelial disease. In some aspects, the epithelial disease is a hyperproliferative epithelial disease selected from the group consisting of psoriasis, cutaneous tumors primary to the skin (basal cell carcinoma, squamous cell carcinoma, melanoma, mycosis fungoides, Bowen's disease), viruses (warts, herpes simplex, condyloma acuminata), premalignant and malignant diseases of the female genital tract (cervix, vagina, vulva) and premalignant and malignant diseases of mucosal tissues (oral, bladder, rectal).

### BRIEF DESCRIPTION OF THE DRAWINGS

FIG. 1 depicts the extent of lesional skin after subcutaneous injection (“SC”) of 1 μmol/g body weight of svL4 (SEQ ID NO:1) or svHIC (SEQ ID NO:4), or topical (“T”) treatment with 1 UM svL4 (SEQ ID NO:1) or svHIC (SEQ ID NO:4). Depilation and a 2-hour treatment with 1% SDS every 3 days was followed by addition of LPS to the daily PBS dressing, which resulted in lesions in the epidermal surface (control or “C”). Sizes of lesions were measured on two 1-cm long sections from each of 4 animals in each group. Horizontal bars indicate mean values for each group. *, P=.022; ***, P=.005. Statistical data were generated with the one-way ANOVA test.

FIGS. 2A and 2B depict LPS-induced eczema causing frequent necrotic lesions within a thick epidermis and a high frequency of neutrophils in the dermis. FIG. 2A shows inclusion of 1 μM svL4 (SEQ ID NO:1) in the LPS-containing dressing resulted in a normal morphology with a thin epidermis, and FIG. 2B demonstrates this same treatment resulted in a dermis that was essentially free of neutrophils. Epidermal thickness was measured at 10 sites along a section of skin for each animal. Dots indicate the mean of each section and the horizontal bars indicate the mean for each group. Depilated skin treated with PBS is indicated with “C”. ****, P=000048 for (a) and 00001 for (b).

FIG. 3 depicts reduction in epidermal thickness by topical treatment with peptides. Peptides (2 μM) were added to the LPS treatment for 14 days. Each dot is the mean of measurements of thickness at 20 sites along two 1-cm long sections stained with anti-Ly6G from each of 5 animals. Dots indicate the mean of each section and the horizontal bars indicate the mean for each group. The depilated LPS-treated control is indicated with “C”. ****, P=.00018 for sVL4 (SEQ ID NO:1);.000056 for svC1 (SEQ ID NO:3); and 0002 for sv6D (SEQ ID NO:2).

FIG. 4 depicts the extent of lesions on skin induced with HDM and SEB for 4 days. Each 2-day application of these allergens was preceded by a 2-hour treatment with 1% SDS. svL4 (SEQ ID NO: 1) (2 M) was applied topically or in combination with 1 μM dexamethasone (“Dex”) for another 5 days. The dressings were replaced every day. Lesional areas were determined on three 1-cm sections from each of 5 animals in each group at the end of the 9-day study. The allergen-treated control group treated with PBS is indicated with “C”. Each section is indicated by a dot. Horizontal bars indicate the mean for each group. *, P=.052; ***, P=.0041.

FIGS. 5A and 5B present the results of assays of svL4 (SEQ ID NO:1), svC1 (SEQ ID NO: 3) and sv6D (SEQ ID NO:2) as substrates for TGM2. The cross-linking reaction was performed in microtiter wells with bound polylysine as acceptor and the enzyme from guinea pig liver. The results shown with svL4 (SEQ ID NO:1) (circles), svC1 (SEQ ID NO:3) (triangles) and SV6D (SEQ ID NO:2) (squares) are representative of the results of three assays for each panel.

FIG. 5A presents the results of the assays performed with enzyme solutions freshly prepared from a lyophilized sample from the supplier. FIG. 5B presents the results of the assays performed after storage of the enzyme solution for 6 days at 5° C. Note: The concentration of the enzyme was 4-times higher in FIG. 5B than in FIG. 5A.

FIG. 6A to 6D depict the structures of tetravalent peptides svL4 (SEQ ID NO:1) (FIG. 6A), sv6D (SEQ ID NO:2) (FIG. 6B), svC1 (SEQ ID NO:3) (FIG. 6C), and svHIC (SEQ ID NO: 4) (FIG. 6D).

FIG. 7 depicts two non-limiting examples of excipient/solvent systems for svL4 (SEQ ID NO: 1).

FIGS. 8A and 8B depicts specific binding of svL4 to CLEC10A. FIG. 8A depicts in silico model of CLEC10A with bound sv6D. The peptide is enclosed in red shading. FIG. 8B shows that CLEC10A was specifically recovered from a lysate of human monocyte-derived dendritic cells with (lane 1) mouse anti-human CLEC10A, which was recovered with magnetic beads coated with Protein A, or (lane 2) biotinylated sv6D, which was recovered with magnetic beads coated with streptavidin C1 and subjected to electrophoresis with a BioAnalyzer (Agilent) with sample buffer containing dithiothreitol. Molecular mass markers are indicated for IgG heavy chain (50 kDa), IgG light chain (25 kDa) and a streptavidin C1 subunit (13.6 kDa). The top band is an instrument marker.

FIGS. 9A and 9B presents a mouse model of pyoderma gangrenosum. FIG. 9A depicts effects of a 2-hour topical treatment with 4% SDS every third day and 50 μg/0.2 mL every day, which caused severe loss of the epidermal and dermal tissues, consistent with ulceration. The majority of the epidermis (E) and dermis (D) are necrotic or absent (black arrow) and covered with a thick serocellular crust (C) composed of necrotic debris and degenerative neutrophils. The treatment caused extensive dermal (D,*) and subcutaneous inflammation (SC, **). FIG. 9B depicts the effect of subcutaneous injection of 1 nmol/g body weight of sv6D every day ameliorated the effects of topical 4% SDS/LPS. Morphology of the skin improved, with significant reduction in crust and necrosis scores. The epidermis (E) is shown recovering but still thickened (white line). Inflammatory cell infiltration is present in both the dermis (D, *) and in the subcutis (SC, **). Subcutaneous injection of sv6D resulted in significantly lower (P=0.050) serocellular crust formation and lower epidermal/dermal necrosis scores. The skin was prepared for histochemical analysis at day 5.

FIGS. 10A and 10B shows that subcutaneous sv6D is an effective treatment for PG-like symptoms. Dorsal mouse skin was extensively disrupted by application of 2% SDS for 2 hours on Day 0 and every third day thereafter. LPS (10 μg) was applied topically every day to the SDS-treated skin. sv6D was injected subcutaneously every day at doses of 1 or 0.1 nmol/g body weight. On Day 14 the skin was prepared for histochemical analysis and stained with hematoxylin and eosin.

### DETAILED DESCRIPTION

As used herein, the verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”.

As used herein, the term “polypeptide” refers to a polymer composed of amino acid residues, related naturally occurring structural variants, and synthetic non-naturally occurring analogs thereof linked via peptide bonds. Synthetic polypeptides may be synthesized, for example, using an automated polypeptide synthesizer. As used herein, the term “protein” typically refers to large polypeptides. As used herein, the term “peptide” typically refers to short polypeptides. Conventional notation is used herein to represent polypeptide sequences: the left-hand end of a polypeptide sequence is the amino-terminus, and the right-hand end of a polypeptide sequence is the carboxyl-terminus.

The term “hydrogel” (also called aquagel) refers to a network of oligomers or polymer chains that are water-insoluble, sometimes found as a colloidal gel in which water is the dispersion medium. Hydrogels are natural or synthetic polymers that show superabsorbent properties (having even over 99% water). Hydrogels also possess a degree of flexibility very similar to natural tissue, due to their significant water content.

In some aspects, the peptides or multivalent structured polypeptides of the present disclosure are embedded in a hydrogel or another matrix. One method for embedding the peptides and polypeptides in a hydrogel is described in U.S. Pat. No.9,155,796, which is hereby incorporated by reference.

The term “GalNAc mimetic” as used herein refers to a peptide mimetic such as a peptide, a modified peptide or any other molecule that biologically mimics N-acetylgalactosamine. Examples of GalNAc mimetics include those identified in U.S. Pat. Nos.8,178,649 and 10,093,712. Additional examples of GalNAc mimetics are svL4 (SEQ ID NO:1) and sv6D (SEQ ID NO: 2).

In certain aspects, the present invention relates to the use of GalNAc and/or a GalNAc mimetic, such as svL4 (SEQ ID NO:1) and sv6D (SEQ ID NO:2), as vehicles to deliver a pay-load to the interior of a cell to achieve a response. In one aspect, the response is a negative response (e.g., induction of apoptosis). In another aspect, the response is a positive (e.g., activation of an immunogenic response). In some embodiments, the GalNAc and/or a GalNAc mimetic is constructed as a multivalent GalNAc structure, of which several have been described in the literature (see, e.g., Zhou et al.2021. Development of triantennary N-acetylgalactosamine conjugates as degraders for extracellular proteins. ACS Central Science 7:499-506).

In certain aspects the peptides and multivalent structured polypeptides described herein are administered together with one or more additional therapeutic agents.

In some aspects, the peptide and the additional therapeutic agents, for example, a topical corticosteroid or a monoclonal antibody are conjugated via a linker. In some embodiments, the peptide and the topical corticosteroid or monoclonal antibody are conjugated by a chemical conjugation. Non-limiting examples of the chemical conjugation include Lysine amide coupling, or Cysteine-based conjugation, etc. As used herein, “Lysine amide coupling” or “Amide coupling” refers to conjugation methods connecting the topical corticosteroid or monoclonal antibody and a solvent accessible epsilon-amino group of lysine residues on the peptide using a linker containing activated carboxylic acid ester. As used herein, “Cysteine-based conjugation” refers to conjugation methods relying on a specific reaction between the sidechain sulfhydryl (—SH) group of a cysteine residues of the peptide and a thiol-reactive functional group installed on the topical corticosteroid or monoclonal antibody. In other embodiments, the peptide and the topical corticosteroid or monoclonal antibody are conjugated by an enzymatic conjugation. Non-limiting examples of the enzymatic conjugation include transpeptidation using sortase, transpeptidation using microbial transglutaminase, or N-Glycan engineering, etc.

In some embodiments, the peptide further comprises a C-terminal cysteine, the topical corticosteroid or monoclonal antibody comprises a sulfhydryl (—SH) group or an iodo-group, and the cysteine is conjugated to the—SH group or the iodo-group. In other embodiments, the peptide further comprises a C-terminal carboxyl group, the topical corticosteroid or monoclonal antibody comprises an amino group, and the peptide and the topical corticosteroid or monoclonal antibody are conjugated with a carbodiimide derivative. In other embodiments, the peptide further comprises a C-terminal amino group, the topical corticosteroid or monoclonal antibody comprises an anhydride or carboxyl group, and the peptide and the topical corticosteroid or monoclonal antibody are conjugated with a carbodiimide derivative. In further embodiments, the peptide and the topical corticosteroid or monoclonal antibody are linked via biotin-avidin interaction.

In certain aspects of the present invention, we describe a topical treatment for atopic dermatitis by application of a multivalent peptide, svL4 (SEQ ID NO:1), that serves as a functional substrate for transglutaminases (TGMs).

In some aspects, the present invention relates to the activity of svL4 (SEQ ID NO:1) and SV6D (SEQ ID NO:2) to bind to the receptor CLEC10A (CD301b in the mouse) that is expressed on dermal macrophages, as described in U.S. Pat. No.10,350,260 and U.S. National Stage of International (PCT) Application No. PCT/US2019/12228, U.S. Patent Publication No. 2021/0069285. The peptides as vehicles induce endocytosis of the receptor, thereby delivering a covalently attached pay-load to the interior of the cell.

In some aspects, the present invention relates to a method of treating neutrophil-driven inflammatory disease in a patient. The neutrophil-driven inflammatory disease may be a respiratory condition or a skin condition.

In some implementations, the patient is administered a multivalent structured polypeptide comprising the therapeutic peptide. In some aspects, the multivalent structured polypeptide comprises at least two copies of the therapeutic peptide. In some embodiments, the multivalent structured polypeptide comprises at least two different therapeutic peptides. In certain embodiments, the multivalent structured polypeptide has a central framework, a linker sequence, and at least two arms. Each arm comprises one therapeutic peptide, and each arm is linked to the central framework via the linker sequence. In certain embodiments, each arm of the multivalent structured polypeptide comprises the same therapeutic peptide. In other embodiments, the arms of the multivalent structured polypeptide do not comprise the same therapeutic peptide. In particular embodiments, the multivalent structured polypeptide has four arms and thus is tetravalent. In some aspects, the linker sequence has a sequence comprising GGGS (SEQ ID NO: 8), GGGSGGGS (SEQ ID NO:9), SSSS (SEQ ID NO:10), or SSSSSSSS (SEQ ID NO:11).

In certain embodiments, the multivalent structured polypeptide is tetravalent. In one exemplary embodiment, the multivalent structured polypeptide is svL4 (SEQ ID NO:1), which comprises four arms and each comprises the therapeutic peptide consisting of VQATQSNQHTPR (SEQ ID NO:5).

In another embodiment, the tetravalent therapeutic peptide consists of VQATQS (SEQ ID NO: 7).

In another embodiment, the tetravalent therapeutic peptide consists of NQHTPR (SEQ ID NO: 6).

In another embodiment, the tetravalent therapeutic peptide consists of NPSHPLSG (SEQ ID NO: 12).

The methods may further comprise administering to the patient a second pharmaceutical intervention for treating neutrophil-driven inflammatory disease, for example, a steroid or a monoclonal antibody. In some aspects, the monoclonal antibody targets an inflammatory cytokine to decrease the activation of inflammatory pathways.

Also described are compositions and kits for treating neutrophil-driven inflammatory disease in a patient. The composition comprises the therapeutic peptide or the multivalent structured polypeptide described herein. In some embodiments, the composition further comprises a second pharmaceutical intervention for treating neutrophil-driven inflammatory disease. For example, the second pharmaceutical intervention is a steroid or a monoclonal antibody. In some aspects, the monoclonal antibody targets an inflammatory cytokine to decrease the activation of inflammatory pathways. The kits comprise instructions teaching the administration of the therapeutic peptide or the multivalent structured polypeptide.

Particularly distressful inflammatory diseases affect the lung and therefore the ability of patients to breathe effectively. Unfortunately, persistent inflammation in respiratory system frequently leads to some adverse diseases such as asthma, COPD, and pulmonary fibrosis. In fact, neutrophil infiltration in the inflamed lung is considered a hallmark of Acute Respiratory Distress Syndrome (ARDS).

Asthma, ARDS, COPD, and viral infections by coronavirus have serious consequences, which has been the motivation for the major effort to identify drugs for treatments. Infiltration of neutrophils into lung tissue has been identified as a major cause of the inflammation in these conditions. A large number of drugs, many approved for clinical use and others under development, have been tested for ARDS. Some of the monoclonal antibodies used for treatment of inflammatory respiratory conditions were approved for other uses such as AD and psoriasis.

Accordingly, in certain implementations, the method of treating neutrophil-driven inflammatory disease in a patient further comprises administering to the subject a second pharmaceutical intervention. In some aspects, the second pharmaceutical intervention is a monoclonal antibody targeting the inflammatory pathways or a leukotriene modifier. Interestingly, corticosteroids are pro-inflammatory in these conditions and are not suitable as therapeutic agents. To improve ease in breathing for the patient, the second pharmaceutical intervention may be a beta agonist, leukotriene modifier, cromolyn sodium, or theophylline.

In particular implementations, the patient is administered the therapeutic peptide or the described composition by inhalation. Accordingly, composition comprising the therapeutic peptide administered to the patient may be aerosolized or in the form of a dry powder. Where the composition comprises the therapeutic peptide in a liquid form, the composition is delivered via a nebulizer so that the therapeutic peptide can be administered by inhalation.

Psoriatic skin is characterized by high expression of IL-17A and IL-17F, which are involved in neutrophil accumulation. Monoclonal antibodies against IL-17A have shown impressive clinical efficacy in about 50% of patients with psoriasis. Clinical studies with an antibody that neutralizes IL-17C, designated MOR106, were abandoned because of futility in treating AD. However, anti-IL-17 antibodies such as secukinumab (Cosentyx, Novartis), ixekizumab (Taltz, Lilly), and brodalumab (Siliq, Ortho Dermatologics) have shown efficacy in roughly half of treated patients. Antibodies against IL-23 have been developed in the past few years to treat psoriasis. Guselkumab (Janssen) and tildrakizumab (Ilumetri, Sun Pharmaceuticals) have been approved by the FDA. Other antibodies, such as ustekinumab (Janssen), risankizumab (Abbvie) and minkizumab (Lilly) are in clinical trials. Most of these antibodies bind subunit p19 of the IL-23 complex. Similar antibodies are being developed, which have the potential of causing significant immune imbalance or impaired response to a danger signal.

For the treatment of skin conditions, the therapeutic peptide or multivalent structured polypeptide is preferably administered topically to an area where the skin condition is believed to be present. In some aspects, particularly when serious ulceration occurs on the skin surface, the therapeutic peptide or multivalent structured polypeptide is administered by subcutaneous injection. In other aspects, the therapeutic peptide or multivalent structured polypeptide is administered locally by topical application. In certain aspects where the therapeutic peptide or multivalent structured polypeptide is applied topically, the peptide is incorporated into a cream, ointment, or lotion. In other aspects, the therapeutic peptide is dissolved into a vehicle excipient mixture for topical application. In certain aspects, the peptide is applied to gauze or a bandage that is placed over an area where the skin condition is believed to be present.

In some aspects, the methods of treating a skin condition associated with a neutrophil-infiltration (for example, AD, eczema, psoriasis or pyoderma gangerosum) further comprise administering to the patient a second pharmaceutical intervention. The second pharmaceutical intervention may be a steroid, quinoline derivatives, macrolides, azathioprine, cyclophosphamide, cyclosporin A, or tricyclic anesthetic compounds, or a drug targeting the inflammatory pathway like a monoclonal antibody, which is used as an existing treatment for the skin conditions. In some aspects, the steroid is a topical corticosteroid, for example, hydrocortisone, triamcinolone, dexamethasone, prednisone and derivatives, triamcinolone acetonide, betamethasone, clobetasol, fluocinonide, fluocinoline. Some second pharmaceutical interventions are topical creams that use a combination of steroids, for example, triamcinolone acetonide and hydrocortisone. Targeted drugs under development include inhibitors of Janus Kinase, in particular JAK1, macrolide-based calcineurin inhibitors (pimecrolimus, Eidel; and tacrolimus, Protopic) that reduce IL-2 production, phosphodiesterase-4 inhibitors (crisaborole, Eucrisa), and monoclonal antibodies (dupilumab, Dupixent, an anti-IL-4 receptor monoclonal antibody (mAb), and tralokinumab, an anti-IL-13 mAb). Dupixent, which binds to the IL-4Ra subunit, inhibits the action of IL-4 and IL-13. The antibodies are delivered by subcutaneous injection and thus also act on other tissues in the body including the lining of the lungs. In one aspect, the therapeutic peptide and the one or more topical corticosteroids are administered concurrently. In other aspects, the therapeutic peptide and the one or more topical corticosteroids are administered sequentially.

The compositions and kits for comprising a skin condition associated with a neutrophil-infiltration comprise the therapeutic peptide or the multivalent structured polypeptide as described herein. In some embodiments, the composition further comprises a second pharmaceutical intervention for treating neutrophil-driven inflammatory disease. For example, the second pharmaceutical intervention is a steroid or a monoclonal antibody. In some aspects, the monoclonal antibody targets an inflammatory cytokine to decrease the activation of inflammatory pathways.

In some embodiments, the multivalent structured polypeptide or peptide is in a pharmaceutical composition with a pharmaceutically or cosmetically acceptable excipient.

In one embodiment of the present disclosure, the pharmaceutically or cosmetically acceptable excipient may be a preservative, a lubricant, a suspending agent, a wetting agent, a thickening agent, a biocompatible solvent, a surfactant, a complexation agent, and any combination thereof.

In one embodiment of the present disclosure, the preservative may include, but not limit to, sodium benzoate, methyl paraben, propyl paraben, and cresols.

In one embodiment of the present disclosure, the lubricant may be metallic stearates which include, but is not limited to, magnesium, calcium and sodium stearates, stearic acid, talc, polyethylene glycols, and soluble salts. In another embodiment of the present disclosure, the salts include sodium chloride or sodium benzoate.

In one embodiment of the present disclosure, the wetting agent may include, but not limit to, glycerol, sorbitol, and polypropylene glycol.

In one embodiment of the present disclosure, the thickening agent may include, but not limit to, SEPINEO P600, SEPINEO DERM, CARBOPOL 980, sodium carboxymethyl cellulose (Na CMC), xanthan gum, hydroxypropyl cellulose (HPC), and polyvinylpyrrolidone K90 (PVP K90).

As used herein, the term “biocompatible” means generation of no significant undesirable host response for the intended utility. For example, biocompatible materials are non-toxic for the intended utility. Thus, for human utility, biocompatible is more preferably non-toxic to humans or human tissues.

In one embodiment of the present disclosure, the biocompatible solvent may be polyethylene glycol, propylene glycol, glycerol, sorbitol, ethanol, dimethyl sulfoxide, N-methyl-2-pyrrolidone (NMP), N,N-dimethylacetamide (DMA), glycofurol, acetone, isopropyl alcohol (IPA), triglyceride, benzyl benzoate, benzyl alcohol, solketal or any combination thereof.

In one embodiment of the present disclosure, the surfactant may be lecithin, macrogol 15 hydroxystearate, polyoxyethylene alkyl ether, polyoxyethylene castor oil, polyoxyethylene sorbitan fatty acid ester, polyoxyethylene stearate, polyoxylglyceride, sorbitan ester, tocopheryl polyethylene glycol succinate (TPGS) or any combination thereof.

In one embodiment of the present disclosure, the complexation agent may be polyvinyl pyrrolidone or cyclodextrin.

Additional aspects of the present invention include:
1. Subcutaneous injection of svHIC (SEQ ID NO:4), a mimetic of sialic acid, the natural ligand for the inhibitory Siglec receptors, heals surface lesions.
2. svC1 (SEQ ID NO:3) and sv6D (SEQ ID NO:2), the N- and C-terminal halves of svL4 (SEQ ID NO:1), respectively, are as effective as svL4 (SEQ ID NO:1) in healing epidermal lesions and restoration of the epidermis to normal thickness.
3. The three peptides are effective in deleting neutrophils from the dermis of inflamed skin.
4. svL4 (SEQ ID NO:1) is a functional substrate of transglutaminase-2, which cross-links the peptide to lysine residues on proteins. The activities with svC1 (SEQ ID NO:3) and sv6D (SEQ ID NO:2) as substrates increase when the enzyme ages during storage for several (6) days.
5. Whereas transglutaminase-2 is short-lived after induction of synthesis in inflamed skin, fresh enzyme, which is active with svL4 (SEQ ID NO:1), and aged enzyme, which is nearly equally active with svL4 (SEQ ID NO:1), svC1 (SEQ ID NO:3) and sv6D (SEQ ID NO: 2), would effectively heal eczema over two weeks of treatment.
6. The combination of low dose glucocorticoid steroids with the peptides would provide an effective therapy as well as the peptides alone.

Subcutaneous injection of sv6D (SEQ ID NO:2) is required in some aspects to engage CLEC10A on macrophages in the dermis to provide a means to treat more serious skin diseases. Neutrophilic dermatoses are a family of cutaneous disorders that result from an exaggerated inflammatory response to injury, which leads to destruction of the epidermis and slow-to-heal ulcers. Prototypic examples are Sweet's syndrome, characterized by painful plaques or nodules and often accompanied by fever, joint pain and headache (Navarini et al., 2016; Agrawal et al., 2022), and pyoderma gangrenosum (PG) (Maverakis et al., 2022). The etiology of Sweet's syndrome is complex and not understood, but the hypersensitivity is mediated by cytokines such as IL-1α/β followed by infiltration of neutrophils (Agrawal et al., 2022). Maverakis et al. (2020) provided an excellent primer on the causes, symptoms and treatment of PG, which is a rare disorder characterized by rapid (over days) expansion of painful ulcers in the skin. Patients are usually in the later half of life, although they can be of any age, and seem predisposed to an exaggerated immune response to mild trauma or a surgical procedure. Keratinocytes at the site of injury produce IL-1α, IL-1β, IL-8, IL-36 and TNFα, which promote a massive infiltration of neutrophils. Maverakis et al. (2020) describe the signaling pathway from LPS/TLR4 to NF-κB, the prime inducer of genes encoding these pro-inflammatory cytokines, and the release of IL-1B and IL-18, as trigger factors for PG. LPS also initiates polarization of macrophages to the pro-inflammatory M1 phenotype, which normally would phagocytize damaged tissue but, in this case, also contributes to the inflammatory crisis. Although neutrophil migration to the site of injury is a normal and primary response to a wound, in PG the invasion causes extensive necrosis of the tissue, often forming an intradermal lesion that splits the epidermis/upper dermis from the lower dermis. The growth of the ulcer gradually slows, but healing may require a year or more.

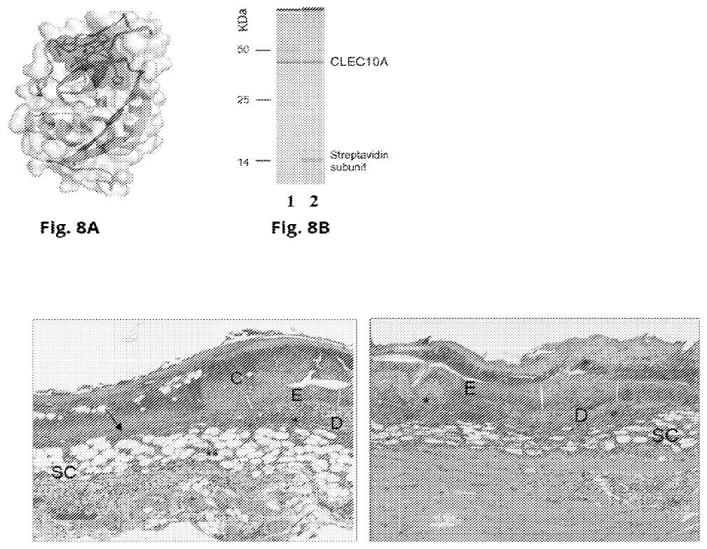

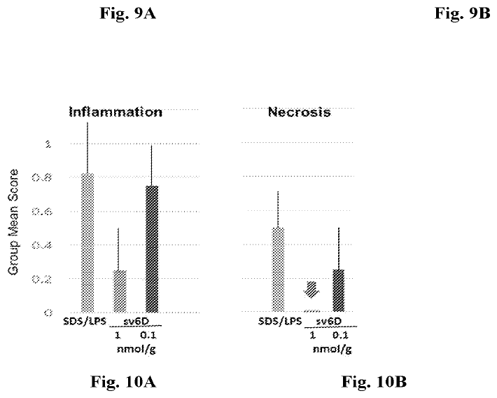

An animal model system has not been established for PG, but we developed a protocol that provides conditions consistent with PG-associated skin ulceration. A 2-hour treatment every second or third day with 4% SDS followed by daily application of 50 μg LPS (0.2 mL) to mouse skin produced a PG-like disruption of the skin within a few days (FIG. 9A). Daily subcutaneous injection of a potent CD301b ligand, sv6D, resulted in significantly lower serocellular crust formation (P=0.050) and lower epidermal/dermal necrosis scores within 5 days (FIG. 9B). After continued treatment for 14 days, inflammation was markedly reduced and necrosis was absent in a dose-dependent manner (FIG. 10). sv6D binds to the receptor CD301/CLEC10A (FIG. 8B), and thus a principal effect of sv6D is thought to be activation of CD301b⁺M2a macrophages. In the mouse, the hypodermal layer is rich in CD301b+M2a macrophages.

CD301/CLEC10A is an endocytic receptor and, at high receptor occupancy, generates an elevated intracellular concentration of Ca²⁺ (Hoober, 2020) M2a macrophages release IL-10 in response to activation of a pathway that leads from an elevated intracellular Ca²⁺ concentration to ERK and CREB, which down-regulates the immune system (Kelly et al., 2010). Macrophages phagocytize neutrophils, which is a prerequisite for resolution of inflammation (Greenlee-Wacker, 2016), and may also form a subdermal barrier to restrict migration of neutrophils into the dermis. Activation of these cells, therefore, would be a major assault on PG and allow healing.

The first line of treatment of neutrophilic dermatoses such as Sweet's syndrome or PG is a high dose of prednisone, which, however, can be given only for a relatively short time and then must be tapered down (Maverakis et al., 2020). Typically, a second type of drug is added, either an inhibitor of calcineurin or Janus kinases or a biologic such as dupilumab. These drugs require several weeks to a month for optimal efficacy, which if not started early will allow a gap in the treatment. In our approach, sv6D acts rapidly to activate macrophages and dendritic cells to eliminate dermal neutrophils and form a barrier to further infiltration of these cells. Thus, a combination of prednisone for the first phase and sv6D for the long-term should be an appropriate treatment for this unfortunate disease.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Experimental Materials and Methods

### Peptides

Synthesis, purification of endotoxin-negative svL4 (SEQ ID NO:1) and sv6D (SEQ ID NO: 2), and their ability to bind CLEC10A were described previously (Eggink et al., 2018).

### Animals

The studies were conducted at Biomodels LLC, an AAALAC accredited facility in Watertown, MA. Approval for this work was obtained from Biomodels IACUC (protocol number 17-0613-4). Female C57BL/6J, 10-week old mice were obtained from The Jackson Laboratory (Bar Harbor, ME) and housed at Biomodels. On day-1, mice were anesthetized with isoflurane and dorsal skin was depilated with electric clippers and chemical treatment with Nair. The skin was then washed with 70% ethanol and PBS. On day 0, mice were again anesthetized and treated for 2 h with 0.2 mL of 1% SDS in a 1 cm² piece of gauze secured with a bio-occlusive dressing (Tegaderm). For naïve mice, sterile water was used instead of SDS. Mice were again anesthetized, the skin was blotted dry, and the skin was covered with gauze containing a mixture of 0.1 mL of 10 μg/mL LPS and 0.1 mL PBS or 2 μM svL4 (SEQ ID NO:1). The treatment solution was replaced every day, whereas the SDS treatment was repeated every three days.

### Induction of Dermatitis

A modification of the mouse model described by Kanemaru et al. (2019) was designed to test activity of the peptides as a treatment for AD.

We found that a 2-hour treatment with 4% SDS every three days, as described by Kanemura et al. (2019), caused extensive damage to the skin. Therefore, 1% SDS, which disrupted the barrier function of human skin (Törmä et al., 2008), was used as a penetration enhancer (Karande et al., 2004). On day-1, dorsal skin from mice was shaved, depilated with thioglycolate and sodium hydroxide (Nair), washed with 70% ethanol followed by a wash with PBS. This treatment caused thickening of the epidermis and extensive infiltration of neutrophils without additional irritants when examined at 14 days. The skin was further treated for 2 h with 1% SDS. The treatment with SDS was repeated every 3 days, with 1-cm² areas of the skin covered with gauze wetted with treatment solution in PBS in the intervening periods.

### Transglutaminase Assay

The reaction was measured in polylysine-coated microtiter wells. The assay (50 μL) contained 100 mM Tris HCl, pH 7.5, 10 mM CaCl₂, 5 mM DTT, 1 mM EDTA, 150 mM NaCl and 0.05% Tween-20. Peptides were added to provide a series of concentrations from 0 to 50 μM. TGM2 (10 μUnits) from guinea pig liver (Sigma-Aldrich, St. Louis, MO) was added to each well, and after 30 min of incubation, the wells were washed 3-times with water. Then 50 μL of 0.1 μg/mL streptavidin conjugated with horseradish peroxidase was added and incubated 20 min. The wells were then washed 4 times with PBS containing 0.05% Tween-20 and 100 μL of 3,3′,5,5′-tetramethylbenzidine substrate were added. The reaction was allowed to proceed 5 min and then stopped with 50 μL 1 N H₂SO₄ and read immediately at 450 nm. An active control peptide with the sequence biotin-TVQQEL was run along with each assay.

### Example 2. Subcutaneous Injection of svHIC (SEQ ID NO:4) Heals Epidermal Lesions

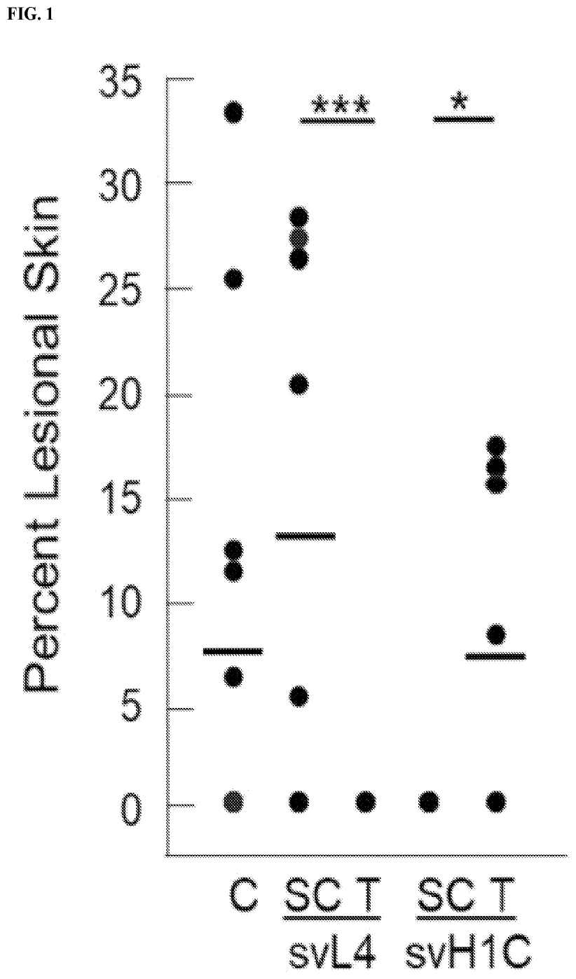

Because we considered lesions an extreme state of eczema, and their closure an essential process in healing, we tested the effects of 1 μM solutions of the peptides when included in the topical application with LPS or administered by subcutaneous injection of 1 nmole/g. The initial analysis quantitated lesional areas on sections of treated skin. Shown in FIG. 1 is the typical variability of the extent of lesions within the groups of animals, which was also a function of the path taken by the section through the tissue. Most sections of skin treated with SDS and LPS contained one or more epidermal lesions. Sections without detectable lesions nevertheless contained a thickened epidermis and the dermis contained numerous neutrophils, which reached a particularly high density under lesions where the basement membrane was lost.

At the end of the treatment period (14 days), lesions were absent when svL4 (SEQ ID NO: 1) was applied topically but were extensive when the peptide was injected subcutaneously. In contrast, svHIC (SEQ ID NO:4) was effective when injected subcutaneously but not when applied topically (see FIG. 1). Although lesions were absent after injections of svHIC (SEQ ID NO:4), most areas of the skin contained a thickened epidermis and abundant neutrophils in the dermis. Thus, subcutaneous injection of svHIC (SEQ ID NO:4), a mimetic of sialic acid, the natural ligand for the inhibitory Siglec receptors, heals surface lesions.

### Example 3. Treatment with svL4 (SEQ ID NO:1), svC1 (SEQ ID NO:3), or sv6D (SEQ ID NO: 2) Alleviates Atopic Dermatitis

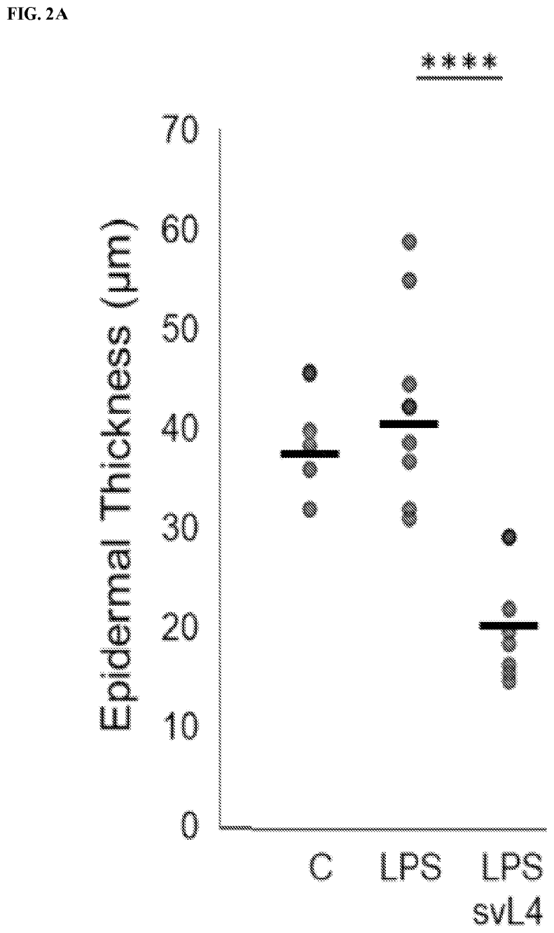

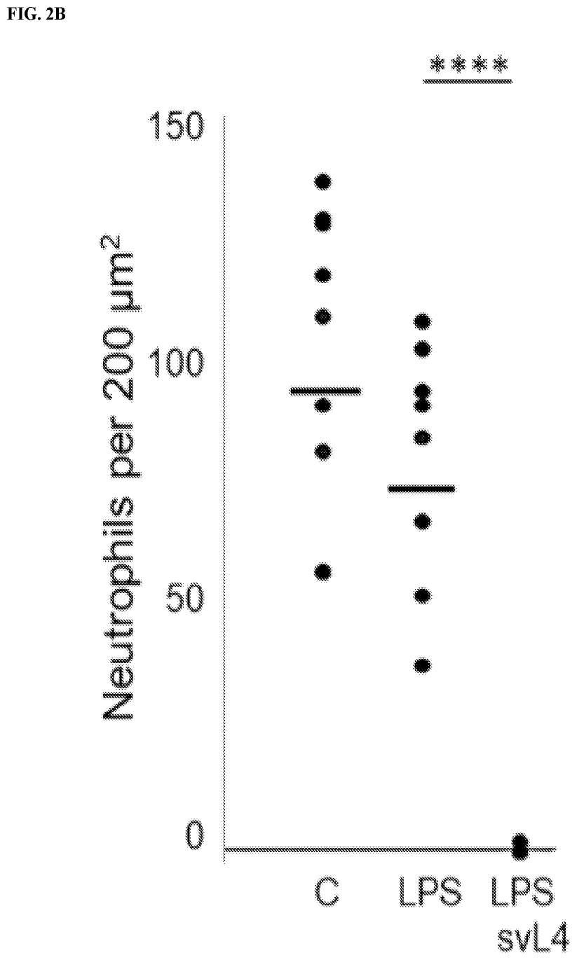

Epidermal necrosis often consisted of a complete loss of the epidermal epithelium. When 1 μM svL4 (SEQ ID NO:1) was applied topically to the skin, the epidermis at the end of the 14-day treatment was uniformly of normal thickness and the dermis was nearly free of neutrophils. Graphical representations of epidermal thickness and neutrophil frequency are presented in FIGS. 2A and 2B.

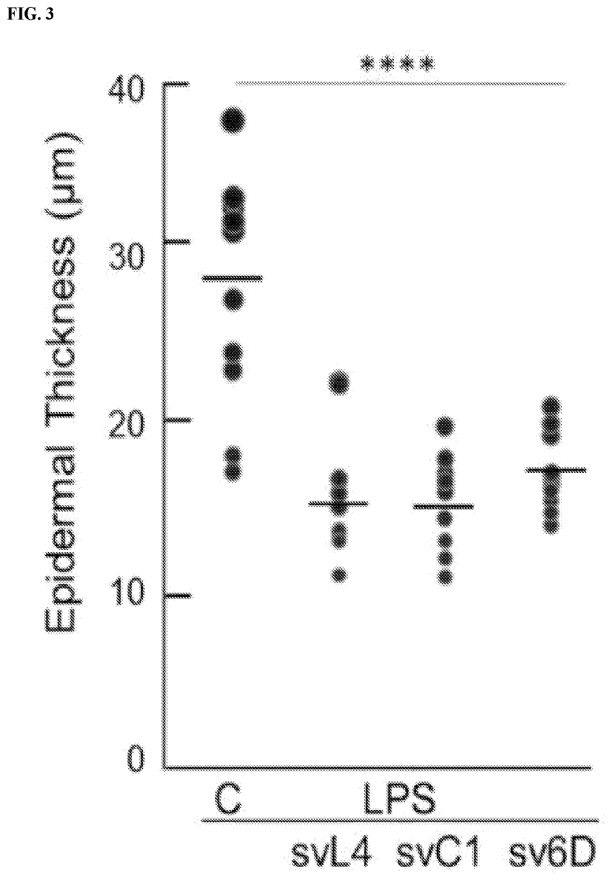

We then asked whether svC1 (SEQ ID NO:3) and sv6D (SEQ ID NO:2) have activity similar to that of svL4 (SEQ ID NO:1) when applied topically to skin along with LPS. In this experiment, the skin was treated with topical 2 μM peptides for 14 days and then sections were stained with anti-Ly6G. As shown in FIG. 3, the three peptides were essentially equally effective in reducing the epidermis to approximately the average normal thickness for murine viable epidermis, excluding the stratum corneum, of 17.5 μm (Wei et al., 2017). Mean values of the thicknesses of peptide-treated animals were 16.1 μm for svL4 (SEQ ID NO:1); 15.5 μm for svC1 (SEQ ID NO:3); and 17.1 μm for sv6D (SEQ ID NO:2). The lack of the GalNAc mimetic sequence in svC1 (SEQ ID NO:3) suggests that targeting of CD301b+ cells is not a major factor in resolving eczema with these peptides. Although peptide svHIC (SEQ ID NO:4) is a mimetic of sialic acid and binds Siglec receptors, it lacks glutamine residues and is not effective when applied topically.

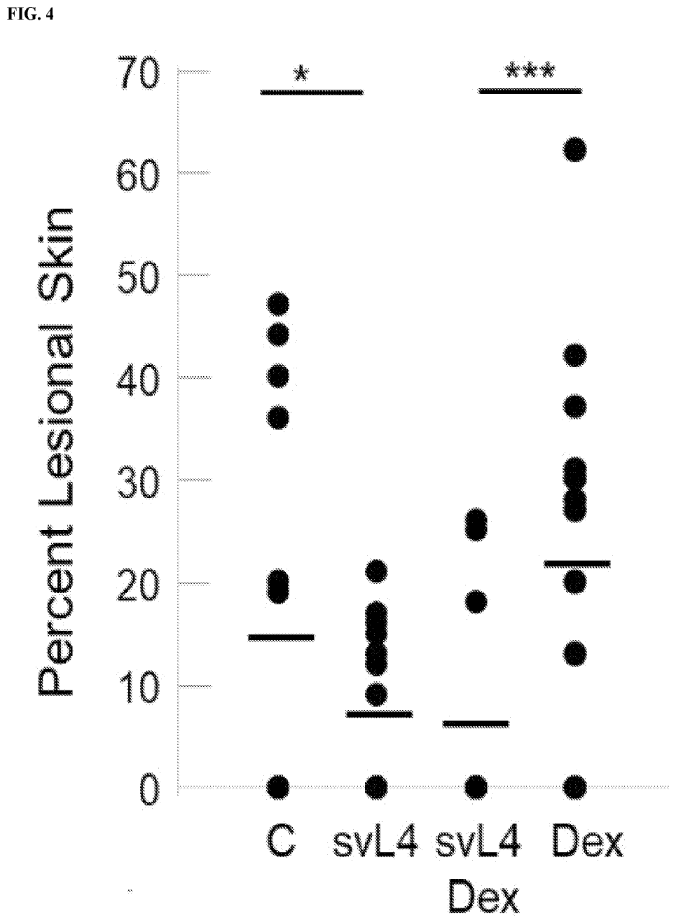

To gain insight into the course of treatment, dermatitis was induced without svL4 (SEQ ID NO: 1) over a period of 4 days with 200 μL of a combination of house dust mite extract (HDM, 10 μg) and Staphylococcal enterotoxin B (SEB, 100 μg), which are commonly encountered allergens (Kawakami et al., 2007). Each 2-day treatment with these allergens was preceded by a 2-hour treatment with 1% SDS, as in previous experiments. After the 4-day treatment with the allergens, the skin was treated with 2 μM svL4 (SEQ ID NO:1)+1 μM dexamethasone for another 5 days, which was expected to provide an intermediate stage in resolution. Similar to the results shown in FIGS. 2A and 2B, in tissue from animals treated with HDM and SEB, thickened epidermis and extensive necrotic lesions with an underlying dense population of neutrophils were evident. Treatment with svL4 (SEQ ID NO:1) reduced epidermal thickness to nearly normal, with areas of thickened as well as thin epidermis but with only a few, small lesions (FIG. 4). Neutrophils were nearly absent in the dermis, particularly underlying the lesions, or at a low frequency at the base of the dermis or subdermal region.

Similar results were obtained with samples from animals treated with svL4 (SEQ ID NO:1) plus dexamethasone (FIG. 4). Treatment with dexamethasone alone showed little improvement over PBS, with several necrotic lesions flanked by areas nearly free of neutrophils. Neutrophils in the dermis under a thickened epidermis were absent or were localized to subdermal tissue in skin treated with svL4 (SEQ ID NO:1) or the combination. Dexamethasone was reported to induce expression of CD301b⁺by monocytes (van Vliet et al., 2006) and cells in the dermis of skin treated with dexamethasone appeared more intensely stained, with a greater abundance of the cells at the base of the dermis. However, treatment with dexamethasone alone showed no improvement over that with PBS (FIG. 4), with an abundance of neutrophils but an absence of CD301b+ cells, underlying lesions (not shown).

### Example 4. Involvement of Transglutaminase (TGM) in Resolution of Dermatitis

sVL4 (SEQ ID NO:1) and sv6D (SEQ ID NO:2) are ligands for CD301b expressed by DCs and macrophages, and CD301b+ cells were abundant during the resolution phase of therapy. The importance of these cells in the final restoration phase is well documented (Shook et al., 2018). However, the initial steps in wound healing are less obvious. We thus considered other possibilities.

Liedén et al. (2012) and Su et al. (2020) demonstrated a remarkable increase in expression of TGMs under the stratum corneum of patients with atopic dermatitis. Because TGM activity is required for formation of a normal epidermal barrier, this upregulation seems to be a response to inflammation and barrier dysfunction. It occurred to us that the glutamine residues in each of the arms of the tetravalent svL4 (SEQ ID NO:1) may provide a substrate for TGMs and thereby offer additional cross-linking opportunities.

TGM1 is expressed in the stratum granulosum of the epidermis and is the major enzyme involved in formation of the cornified envelope beneath the plasma membrane of terminally differentiating keratinocytes (Kalinin et al., 2001). TGM1 initially catalyzes attachment of the scaffold protein involucrin to the inner surface of the membrane followed by cross-linking of the major protein of the cornified envelope, loricrin. Reduction of the cell to a collapsed, insoluble physical barrier is accompanied by replacement of the plasma membrane with ceramide lipids that seal the space between cells.

Whereas TGM1 is confined to the cellular interior, resolution of normal epidermal morphology by topical application of svL4 (SEQ ID NO:1) implies an extracellular reaction that cross-links cells. It is possible that within necrotic lesions the cellular structures are disrupted sufficiently for the peptide to gain access to TGM1. Alternatively, TGM2, which is expressed ubiquitously, is secreted from cells and is involved in cell adhesion and wound healing (Griffin et al., 2002; Eckert et al., 2005). Thus, TGM2 may provide the critical activity in restoring the surface barrier.

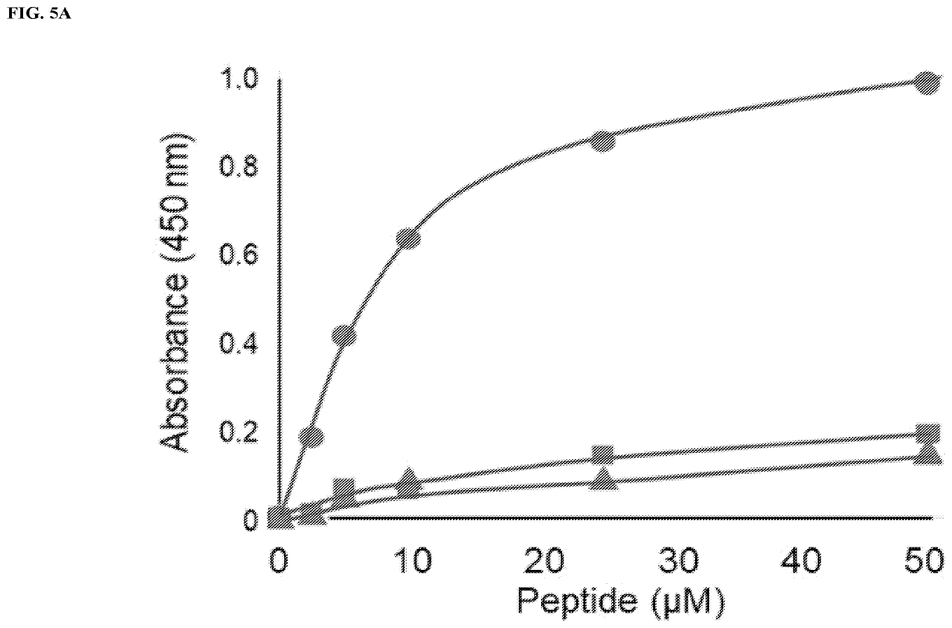

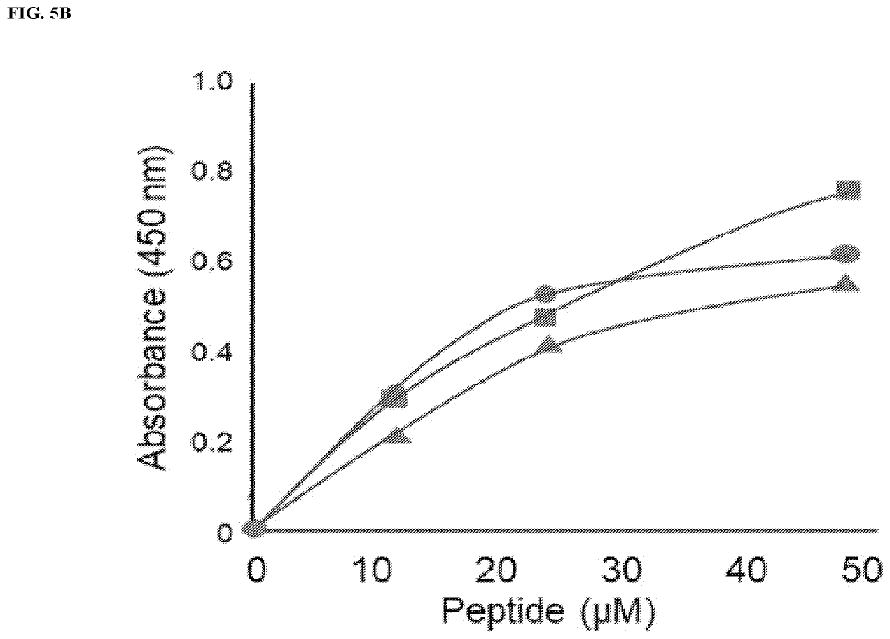

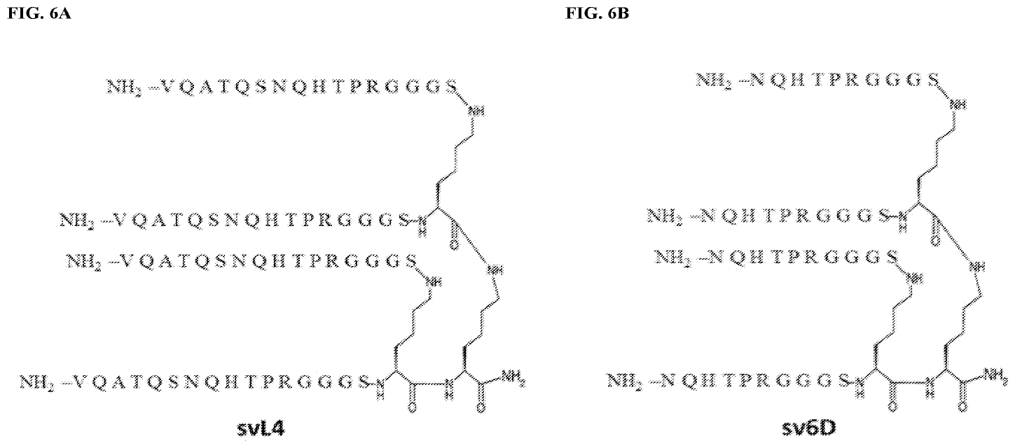

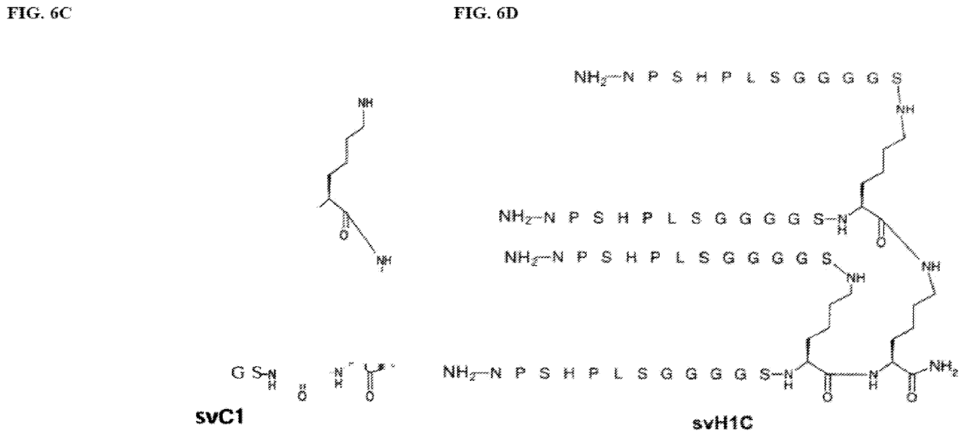

As shown in FIGS. 5A and 5B, the assay with svL4 (SEQ ID NO:1) demonstrated a strong reaction with TGM2. Importantly, as the solution of the enzyme was stored at 5° C. for 6 days, the activity with svL4 (SEQ ID NO:1) declined but that with sv6D (SEQ ID NO:2) and svC1 (SEQ ID NO: 3) increased. Thus, over an extended period of time, newly synthesized as well as aged TGM2 will likely be exposed on the skin surface. Immunohistochemical analysis showed that TGM2 is physically present at the surface of the inflamed and necrotic epidermis.

svL4 (SEQ ID NO:1), svC1 (SEQ ID NO:3) and sv6D (SEQ ID NO:2) provide a TGM2 substrate to enhance cross-linking of the stratum corneum thereby restoring the epidermal barrier and alleviating atopic dermatitis. As shown in Table 1, peptides svC1 (SEQ ID NO:3) and sv6D (SEQ ID NO:2), which are the N- and C-terminal halves of svL4 (SEQ ID NO:1), contain glutamine (Q) at position 2 in the sequence. These Q residues are functional substrates for TGM2 (Pastor et al., 1999; Hitomi et al., 2009). The additional cross-linking ability that the peptides provide allows closure of lesions and repair of the epidermal surface barrier.

TABLE 1
Peptide Sequences

| | | | SEQ |
|---|---|---|---|
| | Identi- | | ID |
| Sequence | fier | MW | NO |
| [(VQATQSNQHTPR-GGGS)₂K₂]K-NH₂ | svL4 | 6,826 | 1 |
| [(NQHTPR -GGGS)₂K₂]K-NH₂ | sv6D | 4,369 | 2 |
| [(VQATQS -GGGS)₂K₂]K-NH₂ | svC1 | 3,893 | 3 |
| [(NPSHPLSG-GGGS)₂K]₂K-NH₂ | SVHIC | 4,592 | 4 |

These data suggest that the mechanism of action of these peptides in the resolution of AD is provision of a substrate for TGMs to enhance cross-linking of the stratum corneum and to restore the epidermal barrier function. Secondarily, the peptide apparently penetrates the epidermis to activate CD301b⁺macrophages in the dermis to eliminate inflammatory neutrophils. svHIC (SEQ ID NO: 4) was not effective when administered topically but stimulated resolution of eczema when injected subcutaneously, presumably by binding to the inhibitory siglec receptors on immune cells and activation of healing mechanisms. Siglecs are expressed by most immune cells and the response to binding of a ligand is a function of the cell and the response of the receptor to the ligand, whether activating or inhibitory (Gonzalez-Gil and Schnaar, 2021).

Sequence-specific peptide substrates have been identified for each of the transglutaminase isozymes 1 to 6 (Sugimura et al., 2008; Fukui et al., 2013; Tanabe et al., 2019). These investigators demonstrated the reaction of the enzyme by covalent iso-peptide linkage between a single peptide and a protein (Tanabe et al., 2019). However, a “single,” monovalent peptide will not provide cross-linking of proteins but will only attach a peptide to one protein. The arms of the multivalent structured polypeptides disclosed herein (e.g., svL4 (SEQ ID NO:1)) provide attachments to and cross-link multiple proteins (i.e., potentially four proteins for a tetravalent structure). A cross-linked mesh is possible only with the disclosed multivalent peptide which serves as a substrate for TGM. Thus, the present invention provides a multivalent peptide that serves as a substrate for TGM cross-linking activity to restore a tight, functional epidermal surface barrier.

Topical application of the peptides svL4 (SEQ ID NO:1), svC1 (SEQ ID NO:3), and sv6D (SEQ ID NO:2) override the response of the skin to the irritants SDS and LPS and allows the return to normal morphology, even in the continuous presence of LPS. The primary response to treatment is the reduction in the number of neutrophils in the dermis. CD301b⁺macrophages, the predominant immune cell in the dermis (Dupasquier et al., 2004), possibly were responsible for phagocytosis of apoptotic neutrophils (Greenlee-Wacker, 2016) during the resolution phase.

### Example 5. Restoration of the Epidermal Barrier Facilitates Formation of a Ca²⁺Gradient and the Return of a Normal Epidermal Morphology

Ca²⁺ plays a major role in regulation of homeostasis of the epidermis. A characteristic Ca²⁺gradient has a peak concentration within the stratum granulosum, with declining concentrations toward the outer stratum corneum and the deeper basal layer (Elias et al., 2002; Mauro et al., 1998). The gradient is composed of extracellular, cytosolic and organelle free Ca²⁺, but only 2% of the stratum granulosum is extracellular space (Celli et al., 2010; Behne et al., 2011). Because the cytosolic Ca²⁺ is usually maintained very low (˜0.1 μM), the average peak concentration of 10 to 20 μM suggests vast stores of Ca²⁺ in cytoplasmic organelles, i.e., endoplasmic reticulum and Golgi structures. The N-terminal region of profilaggrin contains a S100 domain that binds Ca²⁺ (Osawa et al., 2011), which releases the cation as the protein is degraded.

Whereas media with a low Ca²⁺ concentration (30 μM) promotes proliferation of keratinocytes, higher concentrations (>100 μM) are required for differentiation and formation of the outer cell layers during 3-D reconstruction of the epidermis (Bikle et al., 2012; Lee and Lee, 2018; Lee, 2020; Teshima et al., 2020). Disruption of the barrier function of the stratum corneum dissipates the gradient. Thus, expansion of the epidermis upon induction of dermatitis may result from lower Ca²⁺ in the outer layers and a normal, thin epidermis upon treatment likely reflects keratinocyte differentiation upon restoration of the Ca²⁺ gradient. The acidic stratum corneum may serve to attract the basic peptide to the epidermal surface (Behne et al., 2002; Hanson et al., 2002). The observation that topical but not subcutaneous application of svL4 (SEQ ID NO:1) supports a role for the physical presence of the peptide at the outer surface of the skin. Restoration of the barrier function and consequently the Ca²⁺ gradient is a prerequisite to terminal differentiation of keratinocytes and the return to normal morphology of the epidermis (Behne et al., 2011; Elsholz et al., 2014; Lee, 2020).

### Example 6. Induction of Pyoderma Gangrenosum-Like Ulcerative Condition in the Mouse

On Day −1, the hair on the backs of mice was carefully shaven with small animal clippers. Nair was then massaged into the shaved area as to cover the remaining hair completely and reach the skin of the mouse. After 2 minutes, the Nair was removed with sterile gauze and washed thoroughly from the backs/hair using 70% ethanol followed by 2-3 washes with water applied from spray bottle and wiped clean. Mice were patted dry and allowed to recover in home cages. This process was done under anesthesia (isoflurane) with animals remaining on a heating pad throughout.

On Day 0, all animals were again anesthetized with inhaled isoflurane and a 1 cm² square was drawn on the center of the back skin with tissue marker to direct placement of gauze. Animals were then treated with 0.2 ml of a 4% SDS solution applied to a 1 cm² sterile piece of gauze and then secured to the hair-free back skin with a bio-occlusive dressing (Tegaderm) for 2 hours prior to application of Induction 2/Treatment (topical). For the control group, sterile water was used in place of 4% SDS. On Day 2, SDS/water application was repeated 2 hours prior to Induction 2/Treatment (topical).

On Day 0, two hours after Induction 1, animals were again anesthetized with inhaled isoflurane and the Tegaderm and gauze were removed. The skin was blotted dry with sterile gauze. Animals were then treated every day with 0.1 mL of a 0.5 mg/mL solution of LPS in saline and 0.1 ml of saline applied to a 1 cm² sterile piece of gauze secured to the hair-free back skin with a bio-occlusive dressing (Tegaderm). For the control group, only 0.2 mL of saline was applied to the gauze. Animals in separate groups were dosed subcutaneously daily from Day 0 to Day 4 with 1 nmol/g body weight of sv6D. Pathological analysis of histochemical skin sections provided the images for FIGS. 9A and 9B.

In a following experiment, skin was treated for 2 hours every third day with 2% SDS and daily with 10 μg LPS. sv6D was injected subcutaneously every day with doses of 1.0 and 0.1 nmol/g body weight for 14 days. Other details were as described under FIG. 9. Histochemical images were analyzed to provide the graphic data shown in FIGS. 10A and 10B.

### Example 7. Summary of Results

Dermatitis was induced on depilated mouse skin with lipopolysacchride (LPS) after a 2-hour treatment with 1% SDS every third day as a penetration enhancer. The irritants caused thickening of the epidermis, numerous necrotic lesions, and an abundance of neutrophils in the dermis underlying the lesions. When the peptides were included in the topical treatment, neutrophils in the dermis were essentially absent and the skin had returned to its normal morphology after 14 days of treatment (FIGS. 2A and 2B). A 5-day treatment with svL4 (SEQ ID NO: 1) after induction of dermatitis with an extract of house dust mites and Staphylococcal enterotoxin B resulted in partial resolution, with mostly a thin epidermis, only a few small lesions and a low frequency of neutrophils at the base of the dermis or sub-dermally (FIG. 4). svL4 (SEQ ID NO: 1) and sv6D (SEQ ID NO:2), but not svC1 (SEQ ID NO:3), are mimetics of N-acetylgalactosamine and binds to murine C-type lectin receptor MGL2 (CD301b), the ortholog of human CLEC10A, expressed by dendritic cells (DCs) and macrophages in the dermis. The evidence suggests that activated macrophages digest neutrophils. However, an additional feature of the peptides suggests that a mechanism for resolution of AD are glutamine residues in the N-terminal half of each arm of the tetravalent peptide that are substrates for TGM2. Thus, in some aspects, restoration of the epidermal barrier function by activation of TGM2 activity provided by the multifunctional substrates, svL4 (SEQ ID NO:1), svC1 (SEQ ID NO:3) or sv6D (SEQ ID NO: 2) restores epidermal morphology and reduction of neutrophils in the dermis. These data support these peptides as small molecule drugs suitable for clinical use.

Murine skin is used extensively to model treatments of AD (Jin et al., 2009; Martel et al., 2017). Mice express two C-type macrophage galactose-type lectin homologues (Higashi et al., 2002), the galactose (Gal)-specific CD301a (MGL1) that is most strongly expressed in macrophages and CD301b (MGL2), the mouse ortholog of the human N-acetylgalactosamine (GalNAc)-specific C-type lectin receptor CLEC10A (CD301) that is a marker for CD1c⁺DCs (Singh et al., 2009; van Kooyk et al., 2015; Heger et al., 2018; Villani et al., 2017; Brown et al., 2019). Kanemaru et al. (2019) discovered that the NC/Nga strain of mouse has a loss-of-function mutation in the Clec10a gene that encodes MGL1 (CD301a), which leads to susceptibility for AD in response to house dust mites, which contain the primary allergen Der f 2 (Johannessen et al., 2005), a protein that shares a homologous sequence with transglutaminase-3 (TGM3) that serves as an epitope for IgE. Dust mite allergens induced production of proinflammatory cytokines such as IL-6 and TNF-α, a characteristic of dermatitis, which was mediated by toll-like receptor 4 (TLR4). This observation was followed by inducing dermatitis by application of lipopolysaccharide (LPS), a well-known ligand for TLR4. LPS, mediated by CD14, initiates a signaling pathway from TLR4 that leads to activation of NF-κB and release of inflammatory cytokines such as TNF-α, IL-1β, IL-6, IL-8 and IL-12p40 and Ml polarization of macrophages (Lu et al., 2008; Liu et al., 2017). These cytokines are major attractants for neutrophils, which infiltrate the skin at high numbers, particularly in regions where necrotic lesions occur in the epidermis.

We initiated studies to determine whether a small molecule ligand of CD301b would achieve resolution of AD. We tested whether peptides svL4 (SEQ ID NO:1), svC1 (SEQ ID NO:3) and sv6D (SEQ ID NO:2) are effective in reducing inflammation in murine skin induced with LPS.

In our experiments, dorsal skin of mice was shaved, depilated with thioglycolate and sodium hydroxide (Nair™), and washed with 70% ethanol. Treatment with 4% SDS as used by Kanemaru et al. (2019) caused extensive damage to the skin, which led us to treat the skin every third day with 1% SDS for 2 h, a treatment that disrupted the barrier function of human skin (Törmä et al., 2008). Allergens and peptides were applied to a 1-cm² area of depilated skin. At the end of the test period, the skin was excised and sectioned for immunohistochemical analysis to determine the extent of injury and the distribution of specific types of cells. Because we considered lesions an extreme state of eczema, and their closure an essential process in healing, we tested the effects of 1 μM solutions of the peptides when included in the topical application with LPS or administered by subcutaneous injection of 1 nmole/g. The initial analysis quantitated lesional areas on sections of treated skin. Shown in FIG. 1 is the typical variability of the extent of lesions within the groups of animals, which was also a function of the path taken by the section through the tissue. Most sections of skin treated with SDS and LPS contained one or more epidermal lesions. Sections without detectable lesions nevertheless contained a thickened epidermis and the dermis contained numerous neutrophils, which reached a particularly high density under lesions where the basement membrane was lost. At the end of the treatment period (14 days), lesions were absent when svL4 (SEQ ID NO:1) was applied topically but were extensive when the peptide was injected subcutaneously. In contrast, svHIC (SEQ ID NO:4) was effective when injected subcutaneously but not when applied topically. Although lesions were absent after injections of svHIC (SEQ ID NO:4), most areas of the skin contained a thickened epidermis and abundant neutrophils in the dermis (not shown).

Epidermal necrosis often consisted of a complete loss of the epidermal epithelium. When 1 μM svL4 (SEQ ID NO:1) was applied topically to the skin, the epidermis at the end of the 14-day treatment was uniformly of normal thickness and the dermis was nearly free of neutrophils. Graphical representations of epidermal thickness and neutrophil frequency are presented in FIGS. 2A and 2B.

We then asked whether svC1 (SEQ ID NO:3) and sv6D (SEQ ID NO:2) have activity similar to that of svL4 (SEQ ID NO:1) when applied topically to skin along with LPS. In this experiment, the skin was treated with topical 2 μM peptides for 14 days and then sections were stained with anti-Ly6G. As shown in FIG. 3, the three peptides were essentially equally effective in reducing the epidermis to approximately the average normal thickness for murine viable epidermis, excluding the stratum corneum, of 17.5 μm (Wei et al., 2017). Mean values of the thicknesses of peptide-treated animals were 16.1 μm for svL4 (SEQ ID NO:1); 15.5 μm for svC1 (SEQ ID NO:3); and 17.1 μm for sv6D (SEQ ID NO:2). The lack of the GalNAc mimetic sequence in svC1 (SEQ ID NO:3) suggests that targeting of CD301b⁺ cells is not a major factor in resolving eczema with these peptides. Although peptide svHIC (SEQ ID NO:4) is a mimetic of sialic acid and binds siglec receptors, it lacks glutamine residues and also is not effective when applied topically.

Eczema was induced with HDM and SEB to determine whether svL4 (SEQ ID NO:1) is effective against allergens other than LPS, i.e., serve as a general treatment. The allergens were applied to the skin for 4 days, after which the skin was treated with 2 μM svL4 (SEQ ID NO:1) for another 5 days alone or in combination with 1 μM dexamethasone. With analysis after this short period of treatment, we sought to determine how quickly the epidermis was repaired. As shown in FIG. 4, topically applied dexamethasone resulted in no reduction in lesional area from that of untreated skin, but lesions were not detected on most sections treated with svL4 (SEQ ID NO: 1) plus dexamethasone. The efficacy of treatment with svL4 (SEQ ID NO:1) alone was similar to that of the combination of svL4 (SEQ ID NO:1) and dexamethasone. However, this experiment suggested that this combination would be an effective therapy.

Transglutaminase activities are essential for terminal differentiation of the epidermis and formation of the cornified layer. Three isozymes of the Ca²⁺-dependent transglutaminase family, TGM1, TGM3 and TGM5, are expressed in the epidermis and function within terminally differentiating keratinocytes (Eckert et al., 2005). Although confined to the cellular interior, these TGMs could possibly be released from keratinocytes as the result of trauma (Gross et al., 2003). Unlike other members of the family, TGM2 is ubiquitously expressed and is found in the extracellular space (Johnson et al., 1999; Griffin et al., 2002; Chou et al., 2011; Zemskov et al., 2011). TGM2 is expressed in the dermis of healthy skin but may achieve access to and provide the critical activity for repair of the surface barrier in lesional areas (Lorand and Graham, 2003; Verderio et al., 2004; Eckert et al., 2005; Nurminskaya and Belkin, 2012). The observation that topical but not subcutaneous application of svL4 (SEQ ID NO:1) was an effective treatment (FIG. 1) supports a role for the physical presence of the peptide on the surface of the skin and possibly a cross-linking substrate for TGM2.

As shown in FIG. 5A, svL4 (SEQ ID NO:1) is a functional substrate for TGM2 in a reaction that linked svL4 (SEQ ID NO:1) to polylysine. A Kₘ of approximately 7 μM was calculated with svL4 (SEQ ID NO:1)-biotin as a substrate, which compares favorably with the Kₘ of a standard peptide, biotinyl-TVQQEL, of 1 μM (Trigwell et al., 2004). We found that sv6D (SEQ ID NO:2) and svC1 (SEQ ID NO:3) were poor substrates when assayed immediately after dissolving commercial, lyophilized TGM2 in water containing 1 mM EDTA, pH 7.5, and 10 mM DTT. Surprisingly, as the solution of TGM2 aged over several days at 5° C. in this medium, the enzyme lost over half of the activity with svL4 (SEQ ID NO:1) but gained the ability to use sv6D (SEQ ID NO:2) and, to a lesser extent, svC1 (SEQ ID NO:3) as substrates (FIG. 5B). This observation is similar to the increase in cross-linking activity of TGM2 that was found when fibroblasts were irradiated with UVA light (Gross et al., 2003), which suggests that the enzyme unfolds to a less specific conformation over time. The concentration dependence also suggests higher Kₘ values for svC1 (SEQ ID NO:3) and sv6D (SEQ ID NO:2) with the aged enzyme. Haroon et al. (1999) found that TGM2 is short-lived after induction, but with continued inflammation, we expected that a mixture of newly synthesized and aged enzyme would likely be present in the tissues. Whereas sv6D (SEQ ID NO:2) contains the GalNAc mimetic sequence, svC1 (SEQ ID NO:3) lacks this sequence but includes the glutamine residues of svL4 (SEQ ID NO: 1) that are expected to be substrates for TGM2.

The distribution of TGM2 was determined by immunohistochemical analysis of frozen tissue after induction of eczema for 4 days with a combination of house dust mite (HDM) extract (10 μg) and Staphylococcal enterotoxin B (SEB, 100 μg), which are commonly encountered allergens (Kawakami et al., 2007), followed by 5 days of topical treatment with 2 μM svL4 (SEQ ID NO: 1). With this short time of treatment, areas with small lesions and inflamed skin remained. TGM2 was detected under the epidermal/dermal boundary and around hair follicles. In some areas, intense staining with anti-TGM2 was restricted to the dermis in skin with a thickened epidermis. However, where the necrotic epidermis in lesional areas was lost, the layer of tissue containing TGM2 was exposed on the surface. Anti-TGM2 stain was also detected among keratinocytes in areas in which re-epithelialization appeared to occur. Thus, we propose that the primary role of the peptides in resolving lesions is related to their ability to serve as a substrate for TGM2 to enhance cross-linking of structural proteins or cells in the developing stratum corneum.

### Example 8. Formulations of svL4 (SEQ ID NO:1)

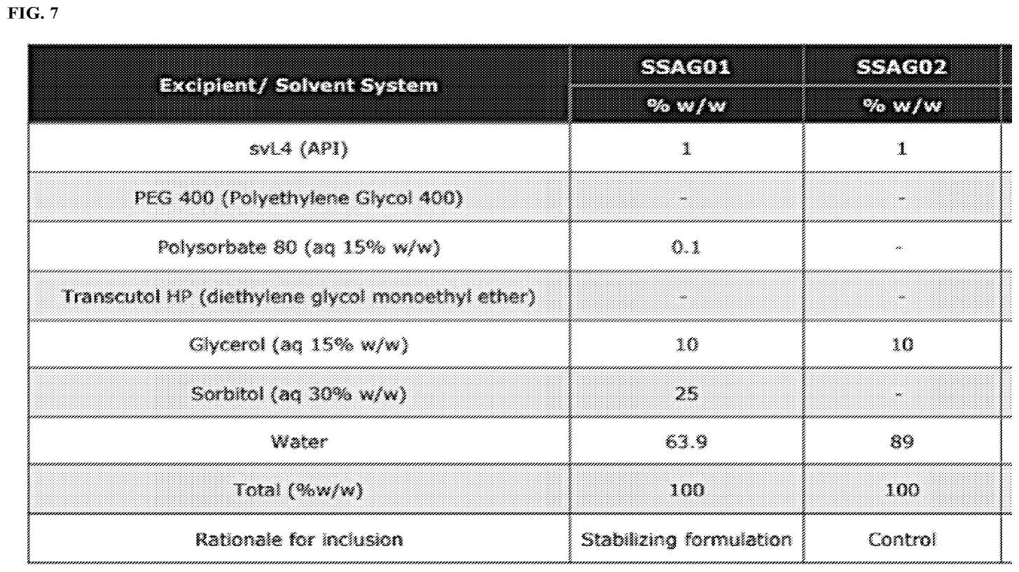

Two different sample formulations of svL4 (SEQ ID NO:1) were developed and evaluated (see FIG. 7). The excipients in the formulations were developed based on the clinical usage of the constituents, solubility, and stability. The SSAGO1 formulation was slightly better than the SSAG02 formulation in terms of solubilizing and stabilizing the peptide. Additional formulations will be evaluated to improve peptide stability and therapeutic efficacy.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

All headings are for the convenience of the reader and should not be used to limit the meaning of the text that follows the heading, unless so specified.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

Agrawal A, Arif S H, Kumarasan K, Janjua D.2022. Current Pediatric Reviews 18:265-273. Behne M J, Meyer J W, Hanson K M, Barry N P, Murata S, Crumrine D, et al. NHE1 regulates the stratum corneum permeability barrier homeostasis. Microenvironment acidification assessed with fluorescence lifetime imagine. J Biol Chem 2002; 277:47399-406.
Behne M J, Sanchez S, Barry N P, Kirschner N, Meyer W, Mauro T M, et al. Major translocation of calcium upon epidermal barrier insult: imaging and quantification via FLIM/Fourier vector analysis. Arch Dermatol Res 2011; 303:103-15.
Bikle D D, Xie Z, Tu C-L. Calcium regulation of keratinocyte differentiation. Expert Rev Endocrinol Metab 2012; 7:461-72.
Bitton A, Avlas S, Reichman H, Itan M, Karo-Atar D, Azouz N P, et al. A key role for IL-13 signaling via the type 2 IL-4 receptor in experimental atopic dermatitis. Sci Immunol 2020 ; 5: eaaw2938.
Brown C C, Gudjonson H, Pritykin Y, Deep D, Lavallée V-P, Mendoza A, et al. Transcriptional basis of mouse and human dendritic cell heterogeneity. Cell 2019; 179:846-63.
Celli A, Sanchez S, Behne M, Hazlett T, Gratton E, Mauro. The epidermal Ca²⁺ gradient: measurement using the phasor representation of fluorescent lifetime imaging. Biophys J 2010 ; 98:911-21.
Chiang C-C, Cheng W-J, Korinek M, Lin C-Y, Hwang T-L. Neutrophils in psoriasis. Front Immunol 2019; 10:2376.
Chou C-Y, Streets A J, Watson P F, Huang L, Verderio EAM, Johnson T S. A crucial sequence for transglutaminase type 2 extracellular trafficking in renal tubular epithelial cells lies in its N-terminal β-sandwich domain. J Biol Chem 2011; 286:27825-35.
Dupasquier M, Stoitzner P, van Oudenaren A, Romani N, Leenen PJM. Macrophages and dendritic cells constitute a major subpopulation of cells in the mouse dermis. J Invest Dermatol 2004; 123:876-9.
Eckert R L, Sturniolo M T, Broome A-M, Ruse M, Rorke E A. Transglutaminase function in epidermis. J Invest Dermatol 2005; 124:481-92.
Eggink L L, Roby K F, Cote R, Hoober J K. An innovative immunotherapeutic strategy for ovarian cancer: CLEC10A and glycomimetic peptides. J ImmunoTher Cancer 2018; 6:28.
Eggink L L, Spyroulias G A, Jones N G, Hanson C V, Hoober J K. A peptide mimetic of 5-acetylneuraminic acid-galactose binds with high avidity to siglecs and NKG2D. PloS One 2015 ; 10: e0130532.
Elias P M, Ahn S K, Brown B E, Crumrine D, Feingold K R. Origin of the epidermal calcium gradient: regulation by barrier status and role of active vs passive mechanisms. J Invest Dermatol 2002; 119:1269-74.
Elsholz F, Harteneck C, Muller W, Friedland K. Calcium—a central regulator of keratinocyte differentiation in health and disease. Eur J Dermatol 2014; 24:650-61.
Fukui M, Kuramoto K, Yamasaki R, Shimizu Y, Itoh M, Kawamoto T, Hitomi K. Identifiation of a highly reactive substrate peptide for transglutaminase 6 and its use in detecting transglutaminase activity in the skin epidermis FEBS J 2013; 280:1420-9.
Gonzalez-Gil A, Schnaar R L. Siglec ligands. Cells 2021; 10:1260.
Greenlee-Wacker M C. Clearance of apopotic neutrophils and resolution of inflammation. Immunol Rev 2016; 273:357-70.
Griffin M, Casadio R, Bergamini C M. Transglutaminases: natures biological glues. Biochem J 2002; 368:377-96.
Gross S R, Balklava Z, Griffin M. Importance of tissue transglutaminase in repair of extracellular matrices and cell death of dermal fibroblasts after exposure to a solarium ultraviolet A source. J Imvest Dermatol 2003; 121:412-23.
Guttman-Yassky E, Nograles K E, Krueger J G. Contrasting pathogenesis of atopic dermatitis and psoriasis Part 1: Clinical and pathologic concepts. J Allergy Clin Immunol 2011; 127:1110-8.
Hamilton J D, Ungar B, Guttman-Yassky E. Drug evaluation review: dupilumab in atopic dermatitis. Immunotherapy 2015; 7:1043-58.
Hanson K M, Behne M J, Barry N P, Mauro T M, Gratton E, Clegg R M. Two-photon fluorescence lifetime imaging of the skin stratum corneum pH gradient. Biophys J 2002; 83:1682-90.
Harb H, Chatila T A. Mechanisms of dupilumab. Clin Exp Allergy 2020; 50:5-14.
Haroon Z A, Hettasch J M, Lai T-S. Dewhirst M W, Greenberg C S. Tissue transglutaminase is expressed, active, and directly involved in rat dermal wound healing and angiogenesis. FASEB J 1999; 13:1787-95.
Heger L, Balk S, Lühr J J, Heidkamp G F, Lehmann CHK, Hatscher L, et al. CLEC10A is a specific marker for human CD1c⁺dendritic cells and enhances their toll-like receptor 7/8-induced cytokine secretion. Front Immunol 2018; 9:744.
Higashi N, Fujioka K, Denda-Nagai K, Hashimoto S, Nagai S, Sato T, et al. The macrophage C-type lectin specific for galactose/N-acetylgalactosamine is an endocytic receptor expressed on monocyte-derived immature dendritic cells. J Biol Chem 2002; 277:20686-93.
Hitomi K, Kitamura M, Sugimura Y. Preferred substrate sequences for transglutaminase 2: screening using a phage-display peptide library. Amino Acids 2009; 36:619-624.
Hoober, J K.2020. ASGR1 and its enigmatic relative, CLEC10A. Int J Mol Sci 21:4818.
Jin H, He R, Oyoshi1 M, Geha R S. Animal models of atopic dermatitis. J Invest Dermatol 2009 ; 129:31-40.
Johannessen B R, Skov L K, Kastrup J S, Kristensen O, Bolwig C, Larsen J N, et al. Structure of the house dust mite allergen Der f 2: implications for function and molecular basis of IgE cross-reactivity. FEBS Lett 2005; 579:1208-12.
Johnson T S, Scholfield C L, Parry J, Griffin M. Induction of tissue transglutaminase by dexamethasone: its correlation to receptor number and transglutaminase-mediated cell death in a series of malignant hamster fibrosarcomas. Biochem J 1998; 331:105-12.
Kalinin A, Marekov L N, Steinert P M. Assembly of the epidermal cornified cell envelope. J Cell Sci 2001; 114:3069-70.
Kamata and Tada.2021. A literature review of real-world effectiveness and safety of dupilumab for atopic dermatitis. J Invest Dermatol Innovations 1:100042.
Kanemaru K, Noguchi E, Tahara-Hanaoka S, Mizuno S, Tateno H, Denda-Nagai K, et al. Clec 10a regulates mite-induced dermatitis. Sci Immunol 2019; 4: eaax6908.
Karande P, Jain A, Mitragotri S. Discovery of transdermal penetration enhancers by high-throughput screening. Nat Biotech 2004; 22:192-7.
Kawakami Y, Yumoto K, Kawakami T. An improved mouse model of atopic dermatitis and suppression of skin lesions by an inhibitor of Tec family kinases. Allergol Internatl 2007; 56:403-9.
Kelly E K, Wang L, Ivashkiv L B.2010. Calcium-activated pathways and oxidative burst mediate zymosan-induced signaling and IL-10 production in human macrophages. J Immunol 184:5545-5552.
Kim T-G, Kim S H, Park J, Choi W, Sohn M, Na H Y, et al. Skin-specific CD301b⁺dermal dendritic cells drive IL-17-mediated psoriasis-like immune response in mice. J Invest Dermatol 2018; 138:844-53.
Kleinman E, Laborada J, Metterle L, Eichenfield L F.2022. What's new in topicals for atopic dermatitis? Am J Clin Dermatol 23:595-603.
Langan S, Irvine A D, Weidinger. Atopic dermatitis. Lancet 2020; 396:345-60.
Lee S E, Lee S H. Skin barrier and calcium. Ann Dermatol 2018; 30:265-75.
Lee A-Y. Molecular mechanism of epidermal barrier dysfunction as primary abnormalities. Int J Mol Sci 2020; 21:1194.
Lieden A, Winge MCG, Sääf A, Kockum I, Ekelund E, Rodriguez E, et al. Genetic variation in the epidermal transglutaminase genes is not associated with atopic dermatitis. PLOS ONE 2012 ; 7: e49694.
Liu T, Zhang L, Joo D, Sun S-C. N F-K B signaling in inflammation. Signal Trans Targ Therapy 2017; 2: e17023.
Lorand L, Graham R M. Transglutaminases: crosslinking enzymes with pleiotropic functions. Nat Rev Mol Cell Biol 2003; 4:140-56.
Lowes M A, Suárez-Fariñas M, Krueger J G. Immunology of psoriasis. Annu Rev Immunol 2014 ; 32:227-55.
Lu Y-C, Yeh W-C, Ohashi P S. LPS/TLR4 signal transduction pathway. Cytokine 2008; 42:145-51. Martel B C, Lovato P, Baumer W, Olivry T. Translational animal models of atopic dermatitis for preclinical studies. Yale J Biol Med 2017; 90:389-402.
Mauro T, Bench G, Sidderas-Haddad E, Feingold K, Elias P, Cullander C. Acute barrier perturbation abolishes the Ca²⁺ and K⁺gradients in murine epidermis: quantitative measurement using PIXE. J Invest Dermatol 1998; 111:1198-201.
Maverakis E, Marzano A V, Le S T, Callen J P, Brüggen M C, Guenova E, Dissemond J, Shinkai K,
Langan S M.2020. Pyoderma gangrenosum. Nature Rev Disease Primers 6:81.
Munera-Campos M, Carrascosa J M. Innovation in atopic dermatitis: from pathogenesis to treatment. Actas Dermasifiliogr 2020; 111:205-21.
Navarini A A, Satoh T K, French L E.2016. Neutrophilic dermatoses and autoinflammatury diseases with skin involvement-innate immune disorders. Semin Immunopathol 38:45-56.
Nurminskaya M V, Belkin A M. Cellular functions of tissue transglutaminase. Int Rev Cell Mol Biol 2012; 294:1-97.
Osawa R, Akiyama M, Shimizu H. Filaggrin gene defects and the risk of developing allergic disorders. Allergol Internatl 2011; 60:1-9.
Pastor M T, Diez A, Pérwz-Payá E, Abad C. Addressing substrate glutamine requirements for tissue transglutaminase using substance P analogues. FEBS Lett 1999:451:231-4.
Saini S, Pansare M. New insights and treatments in atopic dermatitis. Ped Clin North Am 2019 ; 66:1021-33.
Shaw T E, Currie G P, Koudelka C W, Simpson E L. Eczema prevalence in the United States: data from the 2003 National Survey of Children's Health. J Invest Dermatol 2011; 131:67-73.
Shook B A, Wasko R R, Rivera-Gonzalez G C, Salazar-Gatzimas E, López-Giráldez F, Dash B C, et al. Myofibroblast proliferation and heterogeneity are supported by macrophages during skin repair. Science 2018; 362:909.
Singh S K, Streng-Ouwehand I, Litjens M, Weelij D R, Garcia-Vallejo, van Vliet S J, et al. Characterization of murine MGL1 and MGL2 C-type lectins: distinct glycan specificities and tumor binding properties. Molec Immunol 2009; 46:1240-9.
Smith FJD, Irvine A D, Terron-Kwiatkowski A, Sandilands A, Campbell L E, Zhao Y, et al. Los-of-function mutations in the gene encoding filaggrin cause ichthyosis vulgaris. Nat Genet 2006 ; 38:337-341.
Su H, Luo Y, Sun J, Liu X, Ling S, Xu B, et al. Transglutaminase 3 promotes skin inflammation in atopic dermatitis by activating monocyte-derived dendritic cells via D C-SIGN. J Invest Dermatol 2020; 140:170-9.
Sugimura Y, Hosono M, Kitamura M, Tsuda T, Yamanishi K, Maki M, Hitomi K. Identification of preferred substrate sequences for transglutaminase 1-development of a novel peptide that an efficiently detect cross-linking enzuyme activity in the skin. FEBS J 2008; 275:5667-77.
Tanabe Y, Yamane M, Kato M, Teshima H, Kuribayashi M, Tatsukawa H, et al. Studies on differentiation-dependent expresión and activity of distinct transglutaminases by specific substrate peptides using three-dimensional reconstructed epidermis. FEBS J 2019; 286:2536-48.
Teshima H, Kato M, Tatsukawa H, Hitomi K. Analysis of the expression of transglutaminases in the reconstructed human epidermis using a three-dimensional cell culture. Anal Biochem 2020; 603:113606.
Törmä H, Lindberg M, Berne B. Skin barrier disruption by sodium lauryl sulfateexposure alters the expression of involucrin, transglutamnase 1, profilaggrin, and kallikreins during the repair phase in human skin in vivo. J Invest Dermatol 2008; 128:1212-19.
Trigwell S M, Lynch P T, Griffin M, Hargreaves A J, Ronner PLR. An improved colorimetric assay for the measurement of transglutaminase (type II) ε-(γ-glutaminyl) lysine cross-linking activity. Anal Biochem 2004; 330:164-6.
Urban, K, Chu S, Giesey R L, Mehrmal S, Uppal P, Nedley N, Delost G R. The global, regional, and national burden of atopic dermatitis in 195 countries and territories: an ecological study from the Global Burden of Disease Study 2017. JAAD Internat 2021; 2:12-18. van Kooyk Y, Ilarregui J M, van Vliet S J. Novel insights into the immunomodulatory role of the dendritic cell and macrophage-expressed C-type lectin MGL. Immunobiology 2015; 220:185-92 van Vliet S J, van Liempt E, Geijtenbeek TBH, van Kooyk Y. Differential regulation of C-type lectin expression on tolerogenic dendritic cell subsets. Immunobiology 2006; 211:577-585.
Verderio EAM, Johnson T, Griffin M. Tissue transglutaminase in normal and abnormal wound healing: review article. Amino Acids 2004; 26:387-404.
Villani A-C, Saija R, Reynolds G, Sarkizova S, Shekhar K, Fletcher J, et al. Single-cell RNA-seq reveals new types of human blood dendritic cells, monocytes, and progenitors. Science 2017 ; 356: eaah4573.
Wei J C J, Edwards G A, Martin D J, Huang H, Crichton M L, Kendall MAF. Allometric scaling of skin thickness, elasticity, viscoelasticity to mass for micro-medical device translation: from mice, rats, rabbits, pigs to humans. Sci Rep 2017; 7:15885.
Zemskov B A, Mikhailenko I, Hsia R-C, Zaritskaya L, Belkin A M. Unconventional secretion of tissue transglutaminase involves phospholipid-dependent delivery into recycling endosomes. PLOS ONE 2011; 6: e19414.

1. A method of treating a patient having a neutrophil-driven inflammatory disease, the method comprising:
administering to the patient a multivalent structured polypeptide comprising at least one therapeutic peptide;
wherein the sequence of the therapeutic peptide consists of the sequence X₁-X₂-X₃-X₄-X₅-X₆-X₇-X₈-X₉-X₁₀-X₁₁-X₁₂ (SEQ ID NO: 9) with each of X₁, X₂, X₃, X₄, X₅, X₆, X₇, X₈, X₉, X₁₀, X₁₁, and X₁₂ independently being absent or any amino acid residue,
so long as the therapeutic peptide comprises at least 4 amino acid residues and at least one of X₁, X₂, X₃, X₄, X₅, X₆, X₇, X₈, X₉, X₁₀, X₁₁, and X₁₂ is Q.

2. The method of claim 1, wherein the multivalent structured polypeptide has a central framework, a linker sequence, and at least two arms, wherein each arm comprises the therapeutic peptide, and each arm is linked to the central framework via the linker sequence, optionally,
wherein the linker sequence is selected from the group consisting of: GGGS (SEQ ID NO: 8), GGGSGGGS (SEQ ID NO:9), SSSS (SEQ ID NO:10), and SSSSSSSS (SEQ ID NO:11); and/or
wherein the multivalent structured polypeptide is tetravalent.

3. (canceled)

4. The method of claim 1, wherein the at least one therapeutic peptide is VQATQSNQHTPR (SEQ ID NO:5), NQHTPR (SEQ ID NO:6), VQATQS (SEQ ID NO:7), or a combination thereof; or
wherein the multivalent structured polypeptide is svL4 (SEQ ID NO:1), SV6D (SEQ ID NO: 2), or svC1 (SEQ ID NO:3).

5-6. (canceled)

7. The method of claim 1, wherein the at least one therapeutic peptide acts as a substrate for a transglutaminase to induce cross-linking of the stratum corneum, restore the epidermal barrier, and protect the patient from environmental pathogens and allergens; and/or
wherein the multivalent structured polypeptide is effective in deleting neutrophils from the dermis of inflamed skin.

8. (canceled)

9. The method of claim 1, wherein the multivalent structured polypeptide is formulated with a pharmaceutically acceptable excipient selected from the group consisting of a preservative, a lubricant, a suspending agent, a wetting agent, a thickening agent, a biocompatible solvent, a surfactant, a complexation agent, and any combination thereof; and/or
wherein the multivalent structured polypeptide is administered as a topical formulation; and/or
wherein the multivalent structured polypeptide is embedded in a hydrogel.

10-11. (canceled)

12. The method of claim 1, wherein the multivalent structured polypeptide is administered with a topical corticosteroid or monoclonal antibody, optionally,
wherein the at least one topical corticosteroid is selected from the group consisting of triamcinolone acetonide, hydrocortisone, prednisone, and a combination thereof; or
wherein the at least one monoclonal antibody is selected from the group consisting of dupilumab, nemolizumab, secukinumab, and combinations thereof.

13-14. (canceled)

15. The method of claim 12, wherein the multivalent structured polypeptide and topical corticosteroid or monoclonal antibody are conjugated by a chemical conjugation or an enzymatic conjugation, optionally,
wherein the chemical conjugation comprises lysine amide coupling or cysteine-based conjugation, and the enzymatic conjugation comprises transpeptidation using sortase, transpeptidation using microbial transglutaminase, or N-Glycan engineering; or
wherein the multivalent structured polypeptide is conjugated to the topical corticosteroid or monoclonal antibody via a linker comprising an activated carboxylic acid ester; or
wherein the multivalent structured polypeptide and topical corticosteroid or monoclonal antibody are linked with biotin-avidin.

16-18. (canceled)

19. The method of claim 1, further comprising identifying the patient as having a neutrophil-driven inflammatory disease.

20. The method of claim 1, wherein the neutrophil-driven inflammatory disease is atopic dermatitis (AD), psoriasis, or asthma, preferably, wherein the neutrophil-driven inflammatory disease is AD.

21-25. (canceled)

26. The method of claim 1, wherein the multivalent structured polypeptide is svHIC (SEQ ID NO:4) (SEQ ID NO:4).

27-31. (canceled)

32. A pharmaceutical composition comprising:
i) a multivalent structured polypeptide comprising at least one therapeutic peptide;
wherein the sequence of the therapeutic peptide consists of the sequence X₁-X₂-X₃-X₄-X₅-X₆-X₇-X₈-X₉-X₁₀-X₁₁-X₁₂ (SEQ ID NO: 9) with each of X₁, X₂, X₃, X₄, X₅, X₆, X₇, X₈, X₉, X₁₀, X₁₁, and X₁₂ independently being absent or any amino acid residue,
so long as the therapeutic peptide comprises at least 4 amino acid residues and at least one of X₁, X₂, X₃, X₄, X₅, X₆, X₇, X₈, X₉, X₁₀, X₁₁, and X₁₂ is Q; and
ii) either
at least one topical corticosteroid selected from the group consisting of triamcinolone acetonide, hydrocortisone, and a combination thereof; or
at least one monoclonal antibody selected from the group consisting of dupilumab, nemolizumab, secukinumab, and combinations thereof, optionally,
wherein the multivalent structured polypeptide and topical corticosteroid or monoclonal antibody are conjugated by a chemical conjugation or an enzymatic conjugation.

33. The pharmaceutical composition of claim 32, wherein the multivalent structured polypeptide is svL4 (SEQ ID NO:1) (SEQ ID NO:1), sv6D (SEQ ID NO:2) (SEQ ID NO:2), or svC1 (SEQ ID NO:3) (SEQ ID NO:3).

34-35. (canceled)

36. The method of claim 42, wherein the cell is a macrophage or a dendritic cell.

37-39. (canceled)

40. The method of claim 42, wherein the payload is an RNA molecule selected from the group consisting of an mRNA, siRNA, and miRNA; and/or
wherein the conjugate is encapsulated in a lipid-based nanoparticle.

41. (canceled)

42. A method of treating an epithelial disease_or targeting a payload to a cell expressing an endocytic receptor of CD301b (also known as CLEC10A) in a subject in need thereof, the method comprising administering an effective amount of a conjugate to the subject,
wherein the conjugate comprises:
a payload selected from the group consisting of a protein, drug, and RNA molecule; and
N-acetylgalactosamine (GalNAc) or a GalNAc mimetic conjugated to the payload by a chemical conjugation or an enzymatic conjugation.

43. The method of claim 42, wherein a GalNAc mimetic is conjugated to the payload and the GalNAc mimetic is svL4 (SEQ ID NO:1), Sv6D (SEQ ID NO:2), or a combination thereof.

44. The method of claim 42, wherein the epithelial disease is a corneal disease, a conjunctival disease, an oral disease, an epidermal disease, or a hyperproliferative epithelial disease, optionally,
wherein the epithelial disease is a hyperproliferative epithelial disease selected from the group consisting of psoriasis, cutaneous tumors primary to the skin (basal cell carcinoma, squamous cell carcinoma, melanoma, mycosis fungoides, Bowen's disease), viruses (warts, herpes simplex, condyloma acuminata), premalignant and malignant diseases of the female genital tract (cervix, vagina, vulva) and premalignant and malignant diseases of mucosal tissues (oral, bladder, rectal).

45. (canceled)

46. The method of claim 1, wherein the patient has neutrophilic dermatoses, such as pyoderma gangrenosum, comprising subcutaneous injection of SV6D (SEQ ID NO:2).

47. The method of claim 1, wherein the sequence of the therapeutic peptide consists of NPSHPLSG (SEQ ID NO: 12) and the multivalent structured polypeptide has a central framework, a linker sequence, and at least two arms, wherein each arm comprises the therapeutic peptide, and each arm is linked to the central framework via the linker sequence, and
optionally, the linker sequence is selected from the group consisting of: GGGS (SEQ ID NO: 8), GGGSGGGS (SEQ ID NO:9), SSSS (SEQ ID NO:10), and SSSSSSSS (SEQ ID NO:11); and/or the multivalent structured polypeptide is tetravalent.

48. The method of claim 42, further comprising conjugating the payload to N-acetylgalactosamine (GalNAc) and/or a GalNAc mimetic to form a conjugate, wherein the payload is conjugated to the GalNAc and/or the GalNAc mimetic by a chemical conjugation or an enzymatic conjugation.


SEQUENCE LISTING (12 sequences)

SEQ ID NO: 1 - protein, 16 aa, synthetic construct
VQATQSNQHTPRGGGS

SEQ ID NO: 2 - protein, 10 aa, synthetic construct
NQHTPRGGGS

SEQ ID NO: 3 - protein, 10 aa, synthetic construct
VQATQSGGGS

SEQ ID NO: 4 - protein, 12 aa, synthetic construct
NPSHPLSGGGGS

SEQ ID NO: 5 - protein, 12 aa, synthetic construct
VQATQSNQHTPR

SEQ ID NO: 6 - protein, 6 aa, synthetic construct
NQHTPR

SEQ ID NO: 7 - protein, 6 aa, synthetic construct
VQATQS

SEQ ID NO: 8 - protein, 4 aa, synthetic construct
GGGS

SEQ ID NO: 9 - protein, 8 aa, synthetic construct
GGGSGGGS

SEQ ID NO: 10 - protein, 4 aa, synthetic construct
SSSS

SEQ ID NO: 11 - protein, 8 aa, synthetic construct
SSSSSSSS

SEQ ID NO: 12 - protein, 8 aa, synthetic construct
NPSHPLSG